(19) United States
(12) Patent Application Publication
(10) Pub. No.: US 2025/0009856 A1
(43) Pub. Date: Jan. 9, 2025

(54) METHODS FOR TREATING RESPIRATORY DISEASES

(71) Applicant: MUCPharm Pty Ltd, Malvern (AU)

(72) Inventors: David MORRIS, Victoria (AU); Sarah VALLE, Victoria (AU); Ahmed MEKKAWY, Malvern (AU); Krishna PILLAI, Malvern (AU)

(21) Appl. No.: 18/705,481

(22) PCT Filed: Oct. 27, 2022
(86) PCT No.: PCT/AU2022/051294

§ 371 Date: Apr. 26, 2024

(30) Foreign Application Priority Data
Oct. 27, 2021 (AU) 2021903441

(51) Int. Cl. A61K 38/48; A61K 9/00; A61K 45/06; A61P 11/12

(52) U.S. Cl. CPC A61K 38/4873; A61K 9/0073; A61K 45/06; A61P 11/12

(57) ABSTRACT

Disclosed herein is a method for treating a respiratory disease in a patient. The method comprises administering by inhalation a therapeutically effective combination of a glycoprotein affecting protease and a sputum-degrading agent.

### TECHNICAL FIELD

The present invention relates to methods for treating respiratory diseases and to methods for the administration of glycoprotein affecting proteases by inhalation.

### BACKGROUND ART

Respiratory diseases (also commonly referred to as respiratory conditions) affect the respiratory tree, particularly the lungs, and are one of the most common serious medical conditions in the world. Some of the more frequent respiratory diseases are cystic fibrosis, chronic obstructive pulmonary disease (COPD) and asthma. The effects of many respiratory diseases can be managed with treatments that dilate major air passages and improve shortness of breath, thus helping control symptoms and increase the quality of life for people with the disease.

Many respiratory diseases cause the over production of sputum (sometimes called phlegm) in the airways. Sputum generally includes mucins (MUC5B and MUC 5AC, in particular) and cellular materials, and is beneficial in healthy patients because it provides a clearing function where foreign matter such as dust particles, pathogens and other exogenous matter that find their way into the airways become entrained in the sputum and continually cleared by cilia in the air passages. However, sputum can be overproduced when a person's lungs are diseased or damaged, and its consistency can thicken due to it containing elevated levels of components such as proteins, DNA and other cellular materials.

Sputum retention can result in poor oxygenation and an increased risk of bacterial infection, and some therapies therefore promote sputum mobilisation in an attempt to clear the airways. Mucolytic agents are one of many classes of therapeutic agents that have been used to enhance the clearance of stagnating sputum, sometimes in combination with antimicrobial agents when there is infection. Mucolytic agents affect (e.g. by breaking down or otherwise disrupting) the mucin proteins, making them less viscous and hence more easily cleared by the body and/or easier to penetrate with other drugs (e.g. antimicrobial agents).

However, given the sensitivity of the airway tract, and especially the bronchioles and alveoli, only certain therapeutic agents have been approved for administration via inhalation (also referred to as pulmonary administration). It would be advantageous to provide further therapeutic options for treating respiratory diseases, and particularly those which involve overproduction of sputum.

### SUMMARY OF INVENTION

In a first aspect, the present invention provides a method for treating a respiratory disease in a patient. The method comprises administering via inhalation a therapeutically effective combination of a glycoprotein affecting protease and a sputum-degrading agent.

As will be described in further detail below, the inventors have discovered that bromelain, a specific glycoprotein affecting protease, can, in fact, be tolerated when administered via the airways of animal models. Given that bromelain has, to date, only been indicated for topical use for the debridement of severe burn wounds burns on patients' skin, and that it is well known to have serious side effects if administered systemically to a patient (in particular its fibrinolytic action and effect on bleeding), it was extremely surprising to the inventors that the tested animal models tolerated its pulmonary administration. The bronchi and alveoli are extremely sensitive and would not be expected to tolerate such an agent. Indeed, there are numerous published studies that have demonstrated that exposure to cysteine proteases such as papain and bromelain can result in respiratory conditions such as asthma attacks, rhinitis and allergic airway disease.

The inventors believe that the results of their preliminary experiments lead to a reasonable prediction that glycoprotein affecting proteases in addition to bromelain, including those described herein, may also be tolerated when administered via a patient's airways and into their lungs and hence be effective for therapeutic applications such as those described herein. Further experiments, both currently underway and planned, should confirm the inventors' predictions.

In some embodiments, the glycoprotein affecting protease may be a cysteine protease. In some embodiments, the glycoprotein affecting protease may be selected from one or more of the group consisting of: bromelain, papain, ficain, actinidain, zingibain, fastuosain and ananain. Advantages of using bromelain, in particular, will be described below.

In some embodiments, the sputum-degrading agent may be selected from one or more of the group consisting of: a mucolytic agent, a disulphide bond breaking agent, DNase, a PNAG degrading agent, serratiopeptidase, amylase, hyaluronidase and elastase.

In some embodiments, the glycoprotein affecting protease and a sputum-degrading agent may be administered simultaneously or sequentially.

In some embodiments, the respiratory disease may be selected from the group consisting of cystic fibrosis, chronic obstructive pulmonary disease (COPD), acute respiratory distress syndrome (ARDS), bronchiectasis, ventilator associated pneumonia, asthma, sputum retention, mucus plugging, ciliary disfunction such as primary ciliary dyskinesia and bacterial infections.

In some embodiments, the glycoprotein affecting protease and the sputum-degrading agent may be nebulized before administration.

In some embodiments, the glycoprotein affecting protease and sputum-degrading agent are provided in the form of a powder for inhalation from a dry powder inhaler.

In some embodiments, one or more additional therapeutic agents may be co-administered to the patient with the glycoprotein affecting protease and the sputum-degrading agent.

In some embodiments, the disease or condition treated (e.g. respiratory disease) is not a viral infection or a viral respiratory disease such as COVID-19.

In some embodiments, the disease or condition treated (e.g. respiratory disease) is not a bacterial infection involving biofilm.

In a second aspect, the present invention provides a method for treating a respiratory disease in a patient. The method comprises directly instilling a therapeutically effective combination of a glycoprotein affecting protease and a sputum-degrading agent into the patient's airway. The combination may, for example, be directly instilled into the trachea, bronchi or lower airway of the patient. The combination may, for example, be instilled by a direct injection to the site of the disease or a mucus plug in the airway, e.g. at the same time as a bronchoscopy.

The inventors' discovery that bromelain can surprisingly be tolerated when administered via the airways of animal models and their subsequent investigations (some of which are described below) also lead them to predict that the pulmonary administration of glycoprotein affecting proteases may have a systemic therapeutic effect.

In a third aspect therefore, the present invention provides a method for treating a disease or condition in a patient. The method comprises administering a therapeutically effective amount of a glycoprotein affecting protease to the patient via inhalation.

In a fourth aspect, the present invention provides a method for inhibiting cytokine activity in a patient. The method comprises administering via inhalation a therapeutically effective combination of a glycoprotein affecting protease and a sputum-degrading agent.

In a fifth aspect, the present invention provides a method for reducing an inflammatory response in a patient. The method comprises administering via inhalation a therapeutically effective combination of a glycoprotein affecting protease and a sputum-degrading agent.

In a sixth aspect, the present invention provides the use of a combination of a glycoprotein affecting protease and a sputum-degrading agent for treating a respiratory disease in a patient, wherein the combination is administered via inhalation.

In a seventh aspect, the present invention provides the use of a combination of a glycoprotein affecting protease and a sputum-degrading agent for the preparation of an inhalable medicament for treating a respiratory disease in a patient.

In an eighth aspect, the present invention provides an inhalable composition comprising a glycoprotein affecting protease and a sputum-degrading agent for use in treating a respiratory disease in a patient.

In a ninth aspect, the present invention provides the use of a glycoprotein affecting protease for treating a disease or condition in a patient, wherein the combination is administered via inhalation.

In a tenth aspect, the present invention provides the use of a glycoprotein affecting protease for the preparation of an inhalable medicament for treating a disease or condition in a patient.

In an eleventh aspect, the present invention provides an inhalable composition comprising a glycoprotein affecting protease for use in treating a disease or condition in a patient.

In a twelfth aspect, the present invention provides the use of a combination of a glycoprotein affecting protease and a sputum-degrading agent for inhibiting cytokine activity in a patient, wherein the combination is administered via inhalation.

In a thirteenth aspect, the present invention provides the use of a combination of a glycoprotein affecting protease and a sputum-degrading agent for the preparation of an inhalable medicament for inhibiting cytokine activity in a patient.

In a fourteenth aspect, the present invention provides an inhalable composition comprising a glycoprotein affecting protease and a sputum-degrading agent for use in inhibiting cytokine activity in a patient.

In a fifteenth aspect, the present invention provides the use of a combination of a glycoprotein affecting protease and a sputum-degrading agent for reducing an inflammatory response in a patient, wherein the combination is administered via inhalation.

In a sixteenth aspect, the present invention provides the use of a combination of a glycoprotein affecting protease and a sputum-degrading agent for the preparation of an inhalable medicament for reducing an inflammatory response in a patient.

In a seventeenth aspect, the present invention provides an inhalable composition comprising a glycoprotein affecting protease and a sputum-degrading agent for use in reducing an inflammatory response in a patient.

The glycoprotein affecting protease and sputum-degrading agent of the second to seventeenth aspects of the present invention may be as described herein in the context of the first aspect of the present invention. Similarly, the additional features and characterisations described herein in the context of the first aspect of the present invention are generally applicable to the second to seventeenth aspects of the present invention.

In some embodiments of the second to seventeenth aspects of the present invention, the disease or condition treated is not a viral infection or a viral respiratory disease. In some embodiments, the disease or condition treated is not a bacterial infection involving biofilm.

It is to be understood that any features and embodiments described herein in detail in relation to a specific aspect of the invention are equally applicable to other aspects of the invention. Other aspects, features and advantages of the present invention will be described below.

### BRIEF DESCRIPTION OF THE DRAWINGS

FIG. 1 is a graph depicting the results of an Azocasein assay to measure bromelain in samples treated with different concentrations of nebulised BromAc;

FIG. 2 is a graph depicting the results of a NAC assay to measure N-Acetylcysteine (NAC) in mucin samples treated with different concentrations of nebulised BromAc;

FIG. 3 show graphs illustrating the mucolytic effect of BromAc in tracheal aspirate samples from COVID-19 patients using a flow through method, in which the volume of flow through is represented in scatter plots with median and interquartile range (top panel) for a curve with 0, 125 and 250 mcg of bromelain in addition to 2% NAC and, in the bottom panels, connecting line graphs with individual values (n=8; on the right) and mean values (on the left) are displayed;

FIG. 4 shows a correlation of the mucolytic effect of BromAc with tracheal aspirate sample cellularity, in which XY dispersion graphs demonstrate the results for cellularity including total cell count (top panel) and live cell count (bottom panel) according to volume of flow through and the tables display the results of Pearson r coefficient, 95% confidence interval as well as P value and P value summary;

FIG. 5 shows the viscosity assessment after treatment with N-Acetylcysteine (NAC), BromAc 250 mcg and untreated (UN) controls, in which the results are plotted as scatter graphs over floating bars expressing minimum and maximum as well as the average line;

FIG. 6 shows graphs depicting viscosity, shear stress, shear rate as well as torque assessed along the speed of the spindle (in rotations per minute) after treatment with N-Acetylcysteine (NAC-red), BromAc® 250 mcg (blue) and untreated (UN-green) included as controls;

FIG. 7 shows scatter graphs over bars expressing average and standard deviations and demonstrating the effect of BromAc in chemokines present in tracheal aspirate sample from COVID-19 patients;

FIG. 8 shows scatter graphs over bars expressing average and standard deviations and demonstrating the effect of BromAc in pro-inflammatory cytokines present in tracheal aspirate sample from COVID-19 patient;

FIG. 9 shows scatter graphs over bars expressing average and standard deviations and demonstrating the effect of BromAc in growth factors present in tracheal aspirate samples from COVID-19 patients;

FIG. 10 shows scatter graphs over bars expressing average and standard deviations and demonstrating the effect of BromAc in regulatory cytokines present in tracheal aspirate samples from COVID-19 patients;

FIG. 11 shows graphs depicting the effect of N-acetylcysteine on the dynamic viscosity and pipette flow time of artificial and simulated sputum;

FIG. 12 shows graphs depicting the effect of bromelain on the dynamic viscosity and pipette flow time of artificial and simulated sputum;

FIG. 13 shows graphs depicting the effect of BromAc on the dynamic viscosity and pipette flow time of artificial and simulated sputum;

FIG. 14 shows graphs depicting the concentrations of bromelain, N-acetylcysteine and bromelain in artificial and simulated sputum exposed to bromelain, N-acetylcysteine and BromAc;

FIG. 15 shows a graph depicting a comparison of dynamic viscosity to flow speed in artificial and simulated sputa that has been exposed to the agents;

FIG. 16 shows a graph depicting the pipette flow speed of artificial mucin treated with BromAc. DNase and their combination; and

FIG. 17 shows a graph depicting the dynamic viscosity of artificial mucin treated with BromAc, DNase and their combination.

### DETAILED DESCRIPTION OF THE INVENTION

As noted above, in its most general form, the present invention provides a method for treating a disease or condition in a patient, where a therapeutically effective amount of a glycoprotein affecting protease is administered to the patient by inhalation. In other broad forms, the present invention provides therapeutic methods in which a therapeutically effective combination of a glycoprotein affecting protease and a sputum-degrading agent is administered to a patient by inhalation. Alternatively, the combination may be administered to a patient by direct instillation into the trachea or bronchi or lower airway (into the patient's respiratory tract but not via inhalation). In one method, a respiratory disease or condition in a patient is treated. In another method, cytokine activity in a patient is inhibited. In yet another method, an inflammatory response in a patient is reduced.

Diseases and conditions treatable in accordance with the present invention include the respiratory diseases described in further detail below, as well as other diseases and conditions against which the glycoprotein affecting protease has therapeutic effect. Respiratory delivery of the glycoprotein affecting protease may, for example, provide a systemic treatment, including for the brain and sinuses. Diseases and conditions against which the inventors expect that the present invention will have therapeutic effect include cystic fibrosis, chronic obstructive pulmonary disease (COPD), acute respiratory distress syndrome (ARDS), bronchiectasis, ventilator associated pneumonia, asthma, sputum retention, mucus plugging, ciliary disfunction such as primary ciliary dyskinesia and bacterial infections.

As described above, the present invention was made following the inventors' discovery that bromelain can surprisingly be administered, in therapeutically relevant amounts and without adverse effects, via the airway of representative animal models. The amount of bromelain required to achieve a therapeutic effect is an amount which the inventors had expected would cause adverse pulmonary reactions such as asthma, allergic reactions, necrosis or cellular alterations, possibly in addition to adverse issues known to be associated with systemic administration of bromelain (e.g. via injection), such as its fibrinolytic action and effect on bleeding. Surprisingly, however, these issues were not observed in the animal models, potentially enabling a novel delivery mechanism for bromelain and other glycoprotein affecting proteases. The encouraging animal data prompted some of the inventors to self-administer nebulised formulations containing up to 500 μg/ml bromelain and 20 mg/ml N-acetylcysteine three times daily for 10 days, without any adverse side effects being observed.

Some of the inventors of the present application have previously discovered that combinations of proteases such as bromelain and agents such as N-acetylcysteine (already indicated for the treatment of some respiratory diseases) can have a synergistic effect on the dissolution of mucin in the context of cancer therapies. The inventors' current research, some of which is described below, has confirmed that this is also the case for the sputum produced by representative respiratory diseases such as cystic fibrosis. The inventors believe that the results of their preliminary experiments support a reasonable prediction of the therapeutic applications disclosed herein. The inventors also believe that the results of their preliminary experiments support a reasonable prediction that other glycoprotein affecting proteases and sputum-degrading agents will have utility in the present invention. Again, further experiments, both currently underway and planned, should confirm the inventors' predictions.

The term “BromAc”, as used herein, is a combination of bromelain and N-acetylcysteine (NAC), which, as noted above, is a drug combination that has been developed by some of the present inventors for treating mucinous cancers. BromAc was found to rapidly dissolve and remove tumour mucin, whilst neither of the drugs worked alone. BromAc has been shown to remove the mucin protective framework expressed by cancer including MUC1, MUC2, MUC4, MUC5B, MUC5AC and MUC16 due to its effect on glycoproteins and disulphide bonds.

The present invention may be used to treat respiratory diseases, inhibit cytokine activity and/or reduce an inflammatory response in any suitable patient or subject. As would be appreciated, respiratory diseases, cytokine activity and inflammatory response in a patient may be related, and performing the method of the present invention may result on one or more of these effects being achieved. Respiratory diseases which the inventors' data leads them to predict should be treatable in accordance with the present invention include those described above.

In methods where cytokine activity in a patient is inhibited, sputum production would be reduced and conditions such as ARDS would be alleviated, for example. Prevention of hyalin formation may also be reduced, countering the long term effects of ARDS such as pulmonary fibrosis. In methods where an inflammatory response in a patient is reduced, sputum production would be reduced, conditions such as ARDS would be alleviated and better ventilation compliance achieved, for example.

In some embodiments, the patient is a mammalian subject. Typically, the patient will be a human patient, although other subjects may benefit from the present invention. For example, the subject may be a pig, mouse, rat, dog, cat, cow, sheep, horse or any other mammal of social, economic or research importance.

The present invention involves the use of a glycoprotein affecting protease or a combination of a glycoprotein affecting protease and a sputum-degrading agent. Each of these will be described in turn below.

### Glycoprotein Affecting Proteases

Glycoprotein affecting proteases are proteolytic enzymes which cause proteolysis of glycoproteins. Given their preliminary data for bromelain, which is a protease enzyme that affects glycoproteins by hydrolysing glycosidic bonds (as well as other peptide bonds) the glycoproteins, the inventors believe that any glycoprotein affecting protease may be used in the present invention, with routine trial and experimentation being all that would be required (in light of the teachings contained herein) in order to determine any particular glycoprotein affecting protease's suitability. As used herein, the term “Glycoprotein affecting” is to be understood as affecting the glycoprotein (and possibly other peptides) in any therapeutically effective manner such as, for example, by digesting, liquefying or otherwise causing the glycoprotein to disintegrate or degrade. The glycoprotein affecting protease may, for example, be effective to disintegrate glycoproteins. The glycoprotein affecting protease may, for example, be effective to hydrolyse glycosidic bonds of glycoproteins.

The glycoprotein affecting protease may, for example, be a cysteine protease. Cysteine proteases (also known as thiol proteases) degrade proteins via a common catalytic mechanism, and are commonly sourced from fruits including the papaya, pineapple, fig and kiwifruit. Examples of cysteine proteases include bromelain, papain (extracted from papaya) and ananain, a plant cysteine protease in the papain superfamily of cysteine proteases.

There are other plant-derived proteolytic enzymes that express the same characteristics as bromelain and the inventors expect that any plant-derived protease enzymes which have an effect on glycoproteins may be used in the present invention. Again, routine experimentation should be able to confirm the suitability of any particular plant-derived protease enzyme. In some embodiments, for example, the plant-derived protease enzymes may be selected from one or more of the group consisting of Bromelain, Papain (extracted from papaya), Ficain (extracted from figs), Actinidain (extracted from fruits including kiwifruit, pineapple, mango, banana and papaya), Zingibain (extracted from ginger) and Fastuosain (a cysteine proteinase from Bromelia fastuosa). Asparagus, mango and other kiwi fruit and papaya proteases may also be used.

Active fractions of glycoprotein affecting proteases may be used in the present invention, noting that it may not be necessary for all substances in the protease extract to be included, provided that the fraction itself affects the glycoprotein. It is expected that glycoprotein affecting protease enzymes obtained using genetic recombination may also be used in the present invention.

Bromelain is an extract of the pineapple plant (Ananas Comosus) and is a mixture of substances (including different thiol endopeptidases and other components such as phosphatase, glucosidase, peroxidase, cellulase, esterase, and several protease inhibitors) and it may not be necessary for all of these substances to be included in the combination, provided that the fraction of the substances in the combination can at least affect the glycoproteins.

The Bromelain used in the experiments described herein was commercially sourced from Enzybel Group, with any further processing being performed by Mucpharm Pty Ltd.

### Sputum-Degrading Agents

The present invention also includes a sputum degrading agent. Any agent that is therapeutically tolerable and effective to degrade sputum may be used in the present invention.

In some embodiments, for example, the sputum-degrading agent may be selected from one or more of the group consisting of: a disulphide bond breaking agent, a mucolytic agent, a DNase, a PNAG degrading agent, serratiopeptidase, amylase, hyaluronidase and elastase.

Disulphide bond breaking agents are reducing agents that can cleave disulphide bridges in proteins. As the integrity of many mucin proteins are dependent on disulphide bridges, their breakage should result in unfolding of the proteins, thus degrading the sputum.

Disulphide bond breaking agents which the inventors believe may be used in the present invention include acetylcysteine (also known as N-acetylcysteine or NAC), nacystelyn, N-acystelyn, cysteamine, erdosteine, s-carboxymethylcysteine, glutathione, dithiothreitol, mercapto-ethanesulphonate, carbocysteine, dornase alfa, gelsolin, thymosin P4, dextran, dithiobutylamine (DTBA) and heparin.

In some of the proof of concept experiments conducted by the inventors (some of which are described in further detail below), acetylcysteine was used as a sputum degrading agent. Acetylcysteine is an antioxidant with reducing potential in biological systems and is known to cleave disulphide bridges in proteins, and is also a sulphydryl donor which has activity impacts on the protease-potentiates and regenerate. As the integrity of many mucin proteins are dependent on disulphide bridges, the inventors' postulate that their breakage by acetylcysteine will cause unfolding of these proteins, which helps to degrade the sputum.

Advantageously, acetylcysteine is an approved product for use in the treatment of cystic fibrosis and chronic obstructive pulmonary disease. Acetylcysteine is administered via inhalation, either in dosages of 10% or 20% in 4 ml (w/v) up to four times daily. The inventors note that regulatory approvals for medicaments including acetylcysteine may thus be easier to obtain.

Embodiments of the present invention including acetylcysteine are described below in further detail. A person skilled in the art would, however, appreciate that the teachings contained herein could likely be adapted, using routine trials and experiments, for any agent having a sputum degrading effect.

In this regard. DNA and cellular materials can also be a significant component of sputum produced by patients under respiratory distress, and the inventor has demonstrated (described in further detail below) an enhanced effect on sputum when DNA or cell-degrading agents such as DNase are used in the present invention. DNases which the inventors believe may be used in the present invention include Dornase alfa, DNase Type I and DNase Type II. DNase Type I, in particular, has been tested for many clinical applications without major adverse reactions and is approved by the FDA. It has shown positive outcomes in treating cystic fibrosis, asthma, systemic lupus erythematosus and emphysema.

Poly-N-acetylglucosamine (PNAG) is an extracellular polysaccharide that may be present in sputum and the inventor expects that an even further enhanced effect will occur if PNAG-degrading agents such as calcium gluconate, dispersin B and subtilin are included in the present invention.

Other agents having a sputum degrading effect include mucolytic agents, as well as the enzymes serratiopeptidase, amylase, hyaluronidase and elastase.

The relative proportions of the glycoprotein affecting protease and sputum degrading agent in the combination may vary depending on factors such as the kinds of glycoprotein affecting protease and sputum degrading agent, as well as their intended use. Generally speaking, however, the combinations or compositions may include between about 5 μg/mL and about 2 mg/mL of the glycoprotein affecting protease. These amounts are higher than may be needed to achieve a therapeutic effect but the inventors note that only a fraction of drug administered by nebulisation is deposited within the respiratory tract, with losses occurring due to expiration, condensation on tubing and by delivery to the upper airways. In their experiments to date, for example, the inventors have been nebulising up to 1000 μg/mL bromelain because of such losses. As losses of drug may vary between treatment regimens and apparatus, the amounts of glycoprotein affecting protease described below refer to that which will be received into the patient's respiratory tract, such being measurable using routine techniques (an example of which is described below).

Amounts of glycoprotein affecting protease lower than about 5 μg/mL may not be effective and amounts higher than about 1,000 μg/mL would be more likely to cause undesirable side effects. In some embodiments for example, about 5 μg/mL, 10 μg/mL, 15 μg/mL, 20 μg/mL, 30 μg/mL. 40 μg/mL. 50 μg/mL. 60 μg/mL. 80 μg/mL. 100 μg/mL. 150 μg/mL. 200 μg/mL. 250 μg/mL, 300 μg/mL, 350 μg/mL, 400 μg/mL, 450 μg/mL, 500 μg/mL, 550 μg/mL. 600 μg/mL, 650 μg/mL, 700 μg/mL, 750 μg/mL, 800 μg/mL, 850 μg/mL, 900 μg/mL, 950 μg/mL. 1,000 μg/mL of the glycoprotein affecting protease may be administered into the patient's respiratory tract. Ranges falling within the scope of the amounts recited above are also contemplated.

The amount of the sputum degrading agent in the combination will depend primarily on the nature of the agent. In some embodiments for example, the sputum degrading agent may be a disulphide bond breaking agent (e.g. NAC), in which case the combination or composition may include about 0.5%, 1%, 2%, 3%, 5%, 4%, 6%. 7%, 8%, 9%, 10%, 11%, 12%, 13%, 14%, 15%, 16%, 17%, 18%, 19% or 20% (w/v) of the sputum degrading agent. Amounts of NAC less than about 0.5 mg/mL administered into the patient's respiratory tract are unlikely to be effective and amounts higher than about 20 mg/mL would be more likely to cause undesirable side effects. Ranges falling within the scope of the amounts recited above are also contemplated.

In other embodiments for example, the sputum degrading agent may be a DNase, in which case, the combination or composition may include between about 1 μg/mL and about 1000 μg/ml. Unit doses of DNases such as dornase alfa for nebulising to treat CF are 1 mg/ml. Whilst the combination effects described herein are expected to enable the use of lower doses of DNase than those conventionally required, amounts of less than about 1 μg/mL are unlikely to have significant therapeutic effect. In some embodiments for example, about 1 μg/mL, 5 μg/mL, 10 μg/mL, 15 μg/mL, 20 μg/mL, 30 μg/mL, 40 μg/mL, 50 μg/mL, 60 μg/mL, 80 μg/mL, 100 μg/mL, 200 μg/mL, 300 μg/mL, 400 μg/mL, 500 μg/mL, 600 μg/mL, 700 μg/mL, 800 μg/mL, 900 μg/mL or 1 mg/ml of sputum degrading agent in the form of DNase may be administered into the patient's respiratory tract. Ranges falling within the scope of the amounts recited above are also contemplated.

It is envisaged that, for some respiratory diseases and/or patients, repeated treatments may be beneficial (or necessary) in order to complete an effective treatment. It is also envisaged that, for some respiratory diseases and/or patients, combinations of sputum degrading agents (e.g. a combination of NAC and DNase) may be used.

The combination or composition of the glycoprotein affecting protease and sputum-degrading agent may be administered to the patient in any manner that provides the intended therapeutic effect. They may, for example, be simultaneously administered (e.g. in a single composition), sequentially administered (e.g. in separate compositions, one after the other) or separately administered (e.g. in separate compositions, at different times).

The combinations and compositions of the present invention may also include an additional therapeutic agent. Any additional therapeutic agent having an appropriate indication in the context of treating a respiratory infection may be co-administered to the patient. Examples of additional therapeutic agents include antiviral agents, antibacterial agents, bronchodilators and/or expectorants. The additional therapeutic agent may be simultaneously or (more likely) sequentially administered with the glycoprotein affecting protease and/or sputum-degrading agent.

When needed (or beneficial), the quantities of such additional therapeutic agents may be determined on an as-needed basis using no more than routine trials and experimentation.

For example, antibiotic, antiviral, bronchodilators or expectorants that may provide a therapeutic effect in the context of the present invention may be used. Antibiotics which the inventor expects may be useful include aminoglycosides (e.g. gentamicin), cephalosporins, fluoroquinolones, macrolides and penicillin antibiotics (e.g. ampicillin). An antiviral which the inventor expects may be useful is oseltamivir. Bronchodilators which the inventor expects may be useful include salbutamol, a B2 agonist, and anticholinergics. Expectorants which the inventors expect may be useful include guaifenesin, hypertonic saline, nitric oxide and bromhexine.

In compositions including an additional therapeutic agent, the agent may be present in the composition in any amount that produces a beneficial effect. It is within the ability of a person skilled in the art to determine an appropriate quantity of any such additional therapeutic agent. In some embodiments, two or more additional therapeutic agents may provide beneficial effects, especially if their therapeutic effect is via different mechanisms.

Other components may be included in the present invention which do not necessarily have a direct therapeutic effect. For example, hydrating agents such as mannitol may be used to counter any drying effect of the glycoprotein affecting protease or sputum-degrading agent.

### Administration

The glycoprotein affecting protease or combination of glycoprotein affecting protease and sputum-degrading agent may be administered to a patient's airway in any manner that provides the intended therapeutic or prophylactic effect. The combination may, for example, be administered into the lungs of the patient (e.g. after being nebulized). It may, for example, be sprayed into their trachea or bronchi using specialised medical equipment, such as via a bronchoscope. Alternatively (or in addition), the composition may be sprayed into the patient's nose or mouth when they are inhaling. Alternatively (or in addition), the combination may be nebulised and delivered into an atmosphere surrounding a patient such as a closed system tent or other closed-in environmental spaces for treatment.

Nebulisation is a commonly used method for delivering drugs into the respiratory tract. Nebulisers are delivery devices used to administer medication in the form of a mist inhaled into the lungs, and can use oxygen, compressed air or ultrasonic power to break up solutions and suspensions into small aerosol droplets that are inhaled from the mouthpiece of the device.

Another commonly used method for delivering drugs into the respiratory tract is to use a dry powder inhaler or metered dose inhaler. Such inhalers are well known in the art and deliver a specific amount of medication to the lungs, in the form of a short burst of aerosolized medicine that is usually self-administered by the patient via inhalation.

The experiments conducted to date by the inventors have used jet nebulisers, which are nebulisers where gas passes through a nozzle and draws fluid up by the venturi effect, and vibrating mesh nebulisers. Jet nebulisers are inexpensive and widely available. Vibrating mesh nebulisers are also commonly utilised in intensive care units as part of mechanical ventilation. The mesh vibration is due to a piezo electric device. Portable devices are available (e.g., Aerogen ProX). Ultrasonic nebulisers also achieve very small particles. The inventors have specifically examined the delivery of compositions of bromelain and acetylcysteine using multiple devices (e.g., Aerogen ProX Solo, Pari Turbo-Boy, Philips Respironics) and note that the nebulisation of such compositions is not significantly different to 0.9% normal saline.

### Pharmaceutical Compositions

The combination of glycoprotein affecting protease, sputum-degrading agent and optional further agents used in the methods of the present invention may, in some embodiments, be provided in the form of a pharmaceutical composition comprising a pharmaceutically acceptable carrier.

Such a pharmaceutically acceptable carrier will depend on the route of administration of the composition. Liquid form preparations may include solutions, suspensions and emulsions, for example in 0.9% saline for creating aerosols for intranasal or intratracheal delivery. Suitable pharmaceutically acceptable carriers for use in the pharmaceutical compositions of the present invention include physiologically buffered saline, dextrose solutions and Ringer's solution, etc. As noted above, powder formulations for inhalation are also envisaged.

Pharmaceutical compositions suitable for delivery to a patient may be prepared immediately before delivery into the patient's body or may be prepared in advance and stored appropriately beforehand.

The pharmaceutical compositions and medicaments for use in the present invention may comprise a pharmaceutically acceptable carrier, adjuvant, stabiliser, excipient and/or diluent. The carriers, diluents, excipients and adjuvants must be “acceptable” in terms of being compatible with the other ingredients of the composition or medicament and the delivery method, and be generally not deleterious to the recipient thereof.

It will be understood that, where appropriate, some of the components in the combinations or pharmaceutical compositions described herein may be provided in the form of a metabolite, pharmaceutically acceptable salt, solvate or prodrug thereof. “Metabolites” of the components of the invention refer to the intermediates and products of metabolism.

“Pharmaceutically acceptable”, such as pharmaceutically acceptable carrier, excipient, etc., means pharmacologically acceptable and substantially non-toxic to the subject to which the particular compound is administered.

“Pharmaceutically acceptable salt” refers to conventional acid-addition salts or base addition salts that retain the biological effectiveness and properties of the components and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as for example, tetramethylammonium hydroxide. The chemical modification of a pharmaceutical compound (i.e. drug) into a salt is a technique well known to pharmaceutical chemists to obtain improved physical and chemical stability, hygroscopicity, flow ability and solubility of compounds. See, e.g., H. Ansel et. al., Pharmaceutical Dosage Forms and Drug Delivery Systems (6th Ed. 1995) at pp. 196 and 14561457, which is incorporated herein by reference.

“Prodrugs” and “solvates” of some components are also contemplated. The term “prodrug” means a compound (e.g., a drug precursor) that is transformed in vivo to yield the compound required by the invention, or a metabolite, pharmaceutically acceptable salt or solvate thereof. The transformation may occur by various mechanisms (e.g., by metabolic or chemical processes). A discussion of the use of prodrugs is provided by T. Higuchi and W. Stella, “Prodrugs as Novel Delivery Systems.” Vol. 14 of the A.C.S. Symposium Series, and in Bioreversible Carriers in Drug Design, ed. Edward B. Roche. American Pharmaceutical Association and Pergamon Press, 1987.

### Experimental Results

Experiments conducted by the inventors to demonstrate the effect of specific embodiments of the present invention will now be described.

The bromelain used in the experiments described below was manufactured and provided by Mucpharm Pty Ltd (Australia) as a sterile powder. Bromelain was diluted either in phosphate buffered saline (PBS) when used as single agent, or directly in acetylcysteine solution when used as in combination (sometimes referred to as “BromAc” in the examples), to prepare formulations of various concentrations. Acetylcysteine (sometimes referred to as “Ac” in the examples) 200 mg/ml was purchased from Link Pharma (Australia) or manufactured and provided by Mucpharm Pty Ltd. All other reagents were of Analytical grade from Sigma Aldrich, Sydney, Australia.

### Example 1—Nebulisation of BromAc

As described above, nebulisation of liquids is a commonly used method for delivering drugs into the respiratory tract. The inventors have examined the potential pulmonary delivery of BromAc using multiple devices (e.g., Aerogen ProX, Pari Turbo-Boy, Philips Respironics) and found that the nebulisation of BromAc is not significantly different to that of 0.9% normal saline.

Heat generation in some vibrating mesh nebulisers is variable but of concern as Bromelain is heat sensitive, although temperature rises of approximately 10° C. in the chamber over 10 minutes is not likely to have any significant effect on the drug activity. Indeed, the inventors have examined BromAc activity up to 42° C., with no loss. A Fisher & Paykel humidifier in line with a vibrating mesh nebuliser (ventilator circuit) has been used in numerous experiments and has not been found to alter drug efficacy.

### Example 1.1—Particle Size Distribution of BromAc Aerosols Emitted from Pari LC Sprint Nebulizer

The particle size distributions (PSD) of 5 formulations containing bromelain and acetylcysteine at different concentrations were determined. The formulations were aerosolized using a PARI TurboBOY SX compressor, combined with the Pari LC Sprint nebulizer (PARI GmbH, USA). The nebulizer was connected to a USP induction port (throat), and particle size was measured at flow rate of 15 L/min in Spraytec particle sizer (Malvern Instruments, Malvern, UK). Measurements were performed in triplicate. Results are expressed as D10, D50 and D90, indicating the particle diameter at 10, 50 and 90% in the cumulative distribution.

The formulations were prepared in saline (0.9% w/v) and kept at −20° C. prior to analysis. The formulations tested were:
1. Control: Saline (sodium chloride 0.9% w/v)
2. Low Concentration of Ac (8 mg/mL)
3. High Concentration of Ac (60 mg/mL)
4. Brom at 50 μg/mL
5. Combination 1: Low Ac (8 mg/mL)+Brom (50 μg/mL)
6. Combination 2: High Ac (60 mg/mL)+Brom (50 μg/mL)

All formulations were successfully nebulized using the PARI TurboBoy SX, LC Sprinter nebulizer. As can be seen in Table 1, similar particle size distributions were observed for all formulations, with the D50 of all formulations being smaller than 5 μm, which is deemed suitable for aerosol delivery.

TABLE 1
Particle size of all formulations tested via laser
diffraction expressed as D10, D50 and D90.

| | | | | | | | | | NAC + BR | | NAC + BR | |
|---|---|---|---|---|---|---|---|---|---|---|---|---|
| | | | NAC | | NAC | | BR | | (8 mg/mL + | | (60 mg/mL + | |
| | Saline | | (8 mg/mL) | | (60 mg/mL) | | (50 mcg/mL) | | 50 mcg/mL) | | 50 mcg/mL) | |
| | Mean | SD | Mean | SD | Mean | SD | Mean | SD | Mean | SD | Mean | SD |
| D10 | 1.16 | 0.02 | 1.19 | 0.05 | 1.12 | 0.02 | 1.30 | 0.00 | 1.28 | 0.01 | 1.21 | 0.04 |
| D50 | 3.37 | 0.05 | 3.26 | 0.09 | 2.87b | 0.14 | 3.63 | 0.07 | 3.52 | 0.04 | 3.11 | 0.09 |
| D90 | 8.83 | 0.33 | 8.23c | 0.11 | 7.47d | 0.46 | 9.30a | 0.24 | 8.87 | 0.07 | 7.748 | 0.16 |

Means were analysed by 2-way ANOVA and compared to saline control by Tukey's test.
P were significant when smaller than 0.05: a, P < 0.05; b, P < 0.01; c, P < 0.001; d, P < 0.0001

### Example 1.2—Particle Size Distribution of BromAc Aerosols Emitted from Nasal Spray Bottle, MAD Device and Jet Nebuliser InnoSpire Elegance

The following trials were performed to determine the particle size distribution of aerosol's containing 50 and 250 μg/mL bromelain+20 mg/mL acetylcysteine in NaCl solution emitted from a nasal spray bottle Snotty, MAD device and a jet nebuliser InnoSpire Elegance (Philips Respironics). The size distribution of the droplets emitted from each device was measured on a laser diffractometer (Spraytec®, Malvern Panalytical, Malvern, UK). The Spraytec measures real-time, in-situ particle size distribution of the aerosols passing through a laser beam.

The aerosolised droplets were sized with an inhalation cell and at an acquisition frequency of 2.5 kHz. The outlet of each device was positioned 1 cm from the laser measurement zone to minimise evaporation during measurement. A vacuum pump connected to the other end of the inhalation cell was used to remove the aerosols continuously to 1) prevent re-entrainment of droplets into the laser measurement zone; and 2) keep the laser signal transmission>70% to minimise multiple scattering. Signals from detectors 1-7 were excluded to account for beam steering effects. The real and imaginary refractive indices for the droplets were taken to be the same as those for water, which were 1.33 and 0.00, respectively. The refractive index for air was 1.00. These values were deemed appropriate because all measurements showed low residual values (<0.5%). The raw data was processed to yield an averaged volumetric diameter distribution for a period in a given run.

1 mL of solution was loaded to each of the nasal spray bottle and MAD device for a single measurement. The MAD device was attached to a 6 mL syringe, which was chosen as it fitted ideally on the operator's hand to enable the operator to apply consistent pressure comfortably with one hand. Samples from these two devices were measured six times. The nasal spray bottle was primed five to seven times prior to measurements until an even spray was observed. 10 mL of solution was loaded into the reservoir of the jet nebuliser and the measurement was replicated three times. The duration of nebulisation was the time for the whole 10 mL solution to be nebulised. Particle size distributions were expressed as d10 (volume diameter under which 10% of the particles resides), d50 (volume median diameter), d90 (volume diameter under which 90% of the particles resides) and span, which describes the polydispersity of the aerosols. Additionally, the percentages of particles (by volume) with size smaller than 10 and 5 μm of the whole sprays were included. The results are tabulated below.

TABLE 2
Volumetric droplet diameter distributions of bromelain
aerosols emitted from nasal spray bottle.

| | | | | | | |
|---|---|---|---|---|---|---|
| | | | | | <10 | <5 |
| ID | d₁₀ (μm) | d₅₀ (μm) | d₉₀ (μm) | Span | μm (%) | μm (%) |

Bromelain 50 μg/mL

| | | | | | | |
|---|---|---|---|---|---|---|
| Spray 1 | 18.1 | 36.4 | 81.7 | 1.8 | 0.2 | 0 |
| Spray 2 | 18.5 | 35.5 | 73.8 | 1.6 | 0 | 0 |
| Spray 3 | 19.2 | 36.6 | 75.0 | 1.5 | 0 | 0 |
| Spray 4 | 19.0 | 35.2 | 70.6 | 1.5 | 0 | 0 |
| Spray 5 | 16.2 | 39.5 | 100.0 | 2.1 | 2.5 | 0.6 |
| Spray 6 | 16.9 | 42.0 | 102.8 | 2.0 | 2.5 | 0.5 |
| Mean | 18.0 | 37.5 | 83.8 | 1.7 | 0.8 | 0.2 |
| Standard | 1.2 | 2.7 | 13.7 | 0.3 | 1.3 | 0.3 |
| Deviation | | | | | | |

Bromelain 250 μg/mL

| | | | | | | |
|---|---|---|---|---|---|---|
| Spray 1 | 17.8 | 36.7 | 81.3 | 1.7 | 1.6 | 1.1 |
| Spray 2 | 19.9 | 36.0 | 69.2 | 1.4 | 0.4 | 0.4 |
| Spray 3 | 18.7 | 34.5 | 69.2 | 1.5 | 0 | 0 |
| Spray 4 | 17.7 | 31.0 | 61.4 | 1.4 | 0.6 | 0 |
| Spray 5 | 17.4 | 35.0 | 76.3 | 1.3 | 0.4 | 0 |
| Spray 6 | 17.5 | 33.5 | 69.8 | 1.6 | 0.2 | 0 |
| Mean | 18.2 | 34.5 | 71.2 | 1.5 | 0.6 | 0.2 |
| Standard | 1.0 | 2.0 | 6.8 | 0.1 | 0.6 | 0.4 |
| Deviation | | | | | | |

TABLE 3
Volumetric droplet diameter distributions of
bromelain aerosols emitted from MAD device.

| | | | | | | |
|---|---|---|---|---|---|---|
| | | | | | <10 | <5 |
| ID | d₁₀ (μm) | d₅₀ (μm) | d₉₀ (μm) | Span | μm (%) | μm (%) |

Bromelain 50 μg/mL

| | | | | | | |
|---|---|---|---|---|---|---|
| Spray 1 | 56.7 | 137.8 | 393.8 | 2.5 | 0.1 | 0 |
| Spray 2 | 84.8 | 188.6 | 454.3 | 2.0 | 0 | 0 |
| Spray 3 | 103.4 | 255.6 | 609.2 | 2.0 | 0 | 0 |
| Spray 4 | 74.0 | 230.8 | 652.5 | 2.5 | 0.1 | 0 |
| Spray 5 | 56.4 | 157.0 | 615.9 | 3.6 | 0.3 | 0 |
| Spray 6 | 59.6 | 178.6 | 626.9 | 3.2 | 0.3 | 0 |
| Mean | 72.5 | 191.4 | 558.8 | 2.6 | 0.1 | 0.0 |
| Standard | 18.9 | 44.5 | 107.1 | 0.6 | 0.1 | 0.0 |
| Deviation | | | | | | |

Bromelain 250 μg/mL

| | | | | | | |
|---|---|---|---|---|---|---|
| Spray 1 | 62.5 | 159.3 | 584.0 | 3.3 | 0.2 | 0 |
| Spray 2 | 82.3 | 224.1 | 611.6 | 2.4 | 0.1 | 0 |
| Spray 3 | 63.9 | 173.8 | 598.0 | 3.1 | 0.2 | 0 |
| Spray 4 | 72.1 | 196.4 | 600.7 | 2.7 | 0.1 | 0 |
| Spray 5 | 64.4 | 174.1 | 615.5 | 3.2 | 0.2 | 0 |
| Spray 6 | 65.5 | 170.0 | 589.0 | 3.1 | 0.2 | 0 |
| Mean | 68.5 | 183.0 | 599.8 | 2.9 | 0.2 | 0.0 |
| Standard | 7.6 | 23.5 | 12.3 | 0.3 | 0.1 | 0.0 |
| Deviation | | | | | | |

TABLE 4
Volumetric droplet diameter distributions of bromelain
aerosols emitted from jet nebuliser InnoSpire Elegance.

| | | | | | | | |
|---|---|---|---|---|---|---|---|
| | | | | | | | Nebulisation |
| | d₁₀ | d₅₀ | d₉₀ | | <10 μm | <5 μm | time |
| ID | (μm) | (μm) | (μm) | Span | (%) | (%) | (min, sec) |

Bromelain 50 μg/mL

| | | | | | | | |
|---|---|---|---|---|---|---|---|
| Run 1 | 1.7 | 3.8 | 7.6 | 1.6 | 97.0 | 69.0 | 27, 37 |
| Run 2 | 1.7 | 3.7 | 7.7 | 1.6 | 97.0 | 69.0 | 28, 5 |
| Run 3 | 1.7 | 3.8 | 8.0 | 1.7 | 96.2 | 67.1 | 25, 46 |
| Mean | 1.7 | 3.8 | 7.7 | 1.6 | 96.7 | 68.4 | 26.7, 29.3 |
| Standard | 0.0 | 0.0 | 0.2 | 0.0 | 0.5 | 1.1 | 1.53, 21.5 |
| Deviation | | | | | | | |

Bromelain 250 μg/mL

| | | | | | | | |
|---|---|---|---|---|---|---|---|
| Run 1 | 1.7 | 3.6 | 7.5 | 1.6 | 97.4 | 70.5 | 27, 29 |
| Run 2 | 1.7 | 3.7 | 7.6 | 1.6 | 97.0 | 69.6 | 29, 52 |
| Run 3 | 1.6 | 3.5 | 7.2 | 1.6 | 97.7 | 72.9 | 30, 1 |
| Mean | 1.6 | 3.6 | 7.4 | 1.6 | 97.4 | 71.0 | 28.7, 27.3 |
| Standard | 0.0 | 0.1 | 0.2 | 0.0 | 0.3 | 1.7 | 1.53, 25.5 |
| Deviation | | | | | | | |

Table 2 shows that the nasal spray bottle produced consistent droplet size distributions with volumetric median diameter of 37.5±2.7 μm and 34.5±2.0 μm for the 50 μg/mL and 250 μg/mL bromelain concentrations, respectively. Consistent droplets were produced by pressing down on the side of the bottle firmly and quickly.

The MAD device produced the largest droplets among the three devices, with volumetric median diameter of 191.4±44.5 μm and 183.0±23.5 μm for the 50 μg/mL and 250 μg/mL bromelain concentrations, respectively (Table 3). Although consistent pressure was applied by the operator to the best of her ability when pressing the syringe plunger, large variation in the droplet size distribution was observed for both bromelain concentrations.

Table 4 shows that the droplets generated by the jet nebuliser were the smallest with the narrowest distribution. The volumetric median diameter values for the 50 μg/mL and 250 μg/mL bromelain concentrations were 7.7±0.2 μm and 7.4±0.2 μm, respectively. The droplets size distribution was stable over the entire period of nebulisation and the residual volume of solution remaining in the reservoir at the end of each nebulisation was very low (<0.3 mL).

These data demonstrate that therapeutically relevant amounts of BromAc had the same particle size as saline, meaning that they were able to be nebulised into the appropriate size particle to be delivered into a patient's lower airway.

Table 5 summarises the inventors' evaluation of existing systems that appear suitable for the delivery of BromAc to patients' airways.

TABLE 5
Systems for the delivery of BromAc to patients' airways

| | | | Recommended |
|---|---|---|---|
| | | | for Use for |
| Type | Study | Conclusion | lower airway |
| Pari TurboBoy | Particle size of 5 | All formulations were | Yes |
| SX, LC | formulations | successfully aerosolized by | |
| Sprinter | containing | the jet nebulizer Pari | |
| Nebulizer | Acetylcysteine (Ac) | TurboBoy SX, LC Sprinter, | |
| | and bromelain (Brom) | with particle size suitable | |
| | were studied. | for lung delivery and | |
| | | comparable to 0.9% NaCl. | |
| Jet Nebulizer | Determine the aerosol | Droplets generated by the | Yes |
| InnoSpire | droplet size and | nebulizer were small with | |
| Elegance | distribution of 50 and | narrow distribution. The | |
| (Philips | 250, 500, 1000 μg/mL | droplets size distribution | |
| Respironics) | bromelain + 20 mg/mL | was stable over the entire | |
| | acetylcysteine in NaCl | period of nebulization. 96- | |
| | solution emitted | 98% < 10 um and 69-75% < | |
| | | 5 um in BromAc | |
| | | concentrations up to | |
| | | 1000 ug/20 mg/ml. The | |
| | | residual volume of solution | |
| | | remaining in the reservoir at | |
| | | the end of each nebulization | |
| | | was very low (<0.3 ml). | |
| | | Comparable to 0.9% NaCl. | |
| Aerogen ProX | Determine the aerosol | Droplets generated by the | Yes |
| mesh nebulizer | droplet size and | nebulizer were small with | |
| | distribution of 250, | narrow distribution across | |
| | 500, 1000 μg/mL | all concentrations. The | |
| | bromelain + 20 mg/mL | droplets size and | |
| | acetylcysteine in NaCl | distribution were stable over | |
| | vs saline control | the entire period of | |
| | solution emitted | nebulization, 96.3-98.1% < | |
| | | 10 um (saline control | |
| | | 97.7%) and 59.3-60.9% < | |
| | | 5 um (saline control | |
| | | 64.6%). The residual | |
| | | volume of solution | |
| | | remaining in the reservoir at | |
| | | the end of each nebulization | |
| | | was very low (<0.1 ml). | |
| | | Comparable to 0.9% NaCl. | |
| Jet Nebuliser | | Comparable to Phillips | Yes |
| cannister | | Respironics at flow rates of | |
| utilising wall | | at least 6 L/min. | |
| compressor in | | | |
| hospital setting | | | |
| Mucosal | Determine the aerosol | MAD device produced the | Yes |
| Atomization | droplet size of 250, | largest droplets among the | |
| Device (MAD) | 500, 1000 μg/mL | three devices. The large | |
| | bromelain + 20 mg/mL | variation in the droplet size | |
| | acetylcysteine in NaCl | distribution was observed | |
| | vs saline control | for both bromelain | |
| | solution emitted | concentrations. | |
| | | BromAc may be utilized in | |
| | | patients undergoing | |
| | | bronchoscopy as a direct | |
| | | spray down the endotracheal | |
| | | tube or into the bronchus | |
| Nasal spray | | The nasal spray bottle | No |
| bottle (e.g., | | produced consistent droplet | |
| Snotty) | | size distributions. Consistent | |
| | | droplets were produced by | |
| | | pressing down on the side of | |
| | | the bottle firmly and | |
| | | quickly. Probably not | |
| | | suitable for lung use. | |

### Example 1.3—Concentrations of Bromelain and NAC in Mucin Samples Exposed to Nebulised BromAc

In these experiments, a ventilator (Drager Evita XL) was set up in-line with an endotracheal tube connected to a test lung. An artificial sputum (prepared as described in Example 4) was placed within the endotracheal tube causing partial obstruction and BromAc was nebulised into the circuit. A second exposure of BromAc was applied after a period of rest.

The treated samples were numbered thus:
No 65-Retreat BR 250 μg/ml & 2% NAC (10 ml) with BR 250 μg/ml & 2% NAC (10 ML). Both dose humified @37° C. in ET Tube
No 66-Retreat BR 250 μg/ml & 2% NAC (10 ml) with BR 250 μg/ml & 2% NAC (10 ML). Both dose humified @37° C. in ET Tube
No 67-Retreat BR 250 μg/ml & 2% NAC (10 ml) with BR 250 μg/ml & 2% NAC (10 ML). Both dose humified @37° C. into ET Tube
(No Sample 68)
No 69-Retreat BR 250 μg/ml & 2% NAC (16 ml) with BR 250 μg/ml & 2% NAC (10 ML). 2nd dose humified @37° C. in ET Tube
No 70-Retreat BR 250 μg/ml & 2% NAC (10 ml) with BR 250 μg/ml & 2% NAC (10 ML). 2nd dose humified @37° C. into ET Tube
No 71-Retreat 10 ml Saline with 10 ml saline in ET Tube on Feb. 10, 2021. 2nd dose humidified @37 C
No 72-Retreat BR 250 μg/ml & 2% NAC (20 ml) with BR 250 μg/ml & 2% NAC (10 ML). 2nd dose humified @37° C. in ET Tube
No 73-Retreat BR 500 μg/ml & 4% NAC (10 ml) with BR 250 μg/ml & 2% NAC (10 ML). 2nd dose humified @37° C. in ET Tube

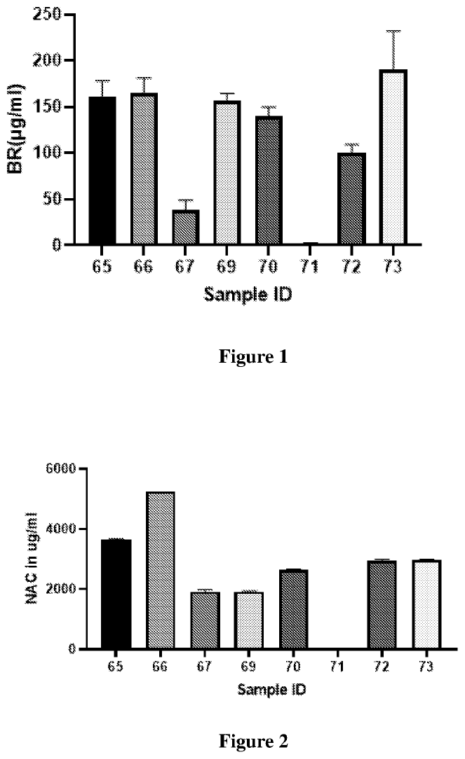

The results of these experiments are shown in FIGS. 1 and 2. These data indicate that bromelain and NAC both accumulate in mucin samples exposed to nebulised BromAc. The accumulated concentrations are within the range expected to produce a mucin degrading effect within the sputum.

### Example 2—Animal Safety Studies

A number of pilot studies were conducted in order to test the safety of administration of Bromelain, with or without acetylcysteine, as an aerosol in rabbit and rodent models.

### Example 2.1—Pilot Study: Safety of Inhaled Bromelain in Rabbits

In this pilot study. 5 New Zealand white rabbits (Pipers farms, NSW, AU) were used to test the safety of administration of bromelain as aerosol. Anaesthesia was induced using a mixture of Acepromazine 1 mg/kg SC+Medetomidine 0.25 mg/kg SC and was maintained during the treatment by inhalation of isoflurane 1-3% in Oxygen at a rate of 1 L/min. Rabbits were administered pre-operative inhalation of Salbutamol 800 micrograms/Kg (Ventolin inhaler, cat #AUST R 317221, GlaxoSmithKline plc., UK) as a bronchodilator to combat potential bronchoconstriction effects of the drug. Ten minutes waiting period was kept between Salbutamol administration and treatment. Each rabbit received one cumulative dose of bromelain using a nebulizer and a v-gel supraglottal airway device as below. The pilot study was divided into 2 experiments as follows:

Experiment 1: Two rabbits were used in the first bromelain aerosol administration experiment. The first rabbit received an aerosol of physiological saline solution (0.9% NaCl), the second rabbit received aerosolized bromelain 0.1 mg/mL (accumulative dose of 0.19 mg in 1.9 mL saline), over 16 min inhalation period (Table 1).

Experiment 2: Three rabbits were used in the second bromelain aerosol administration experiment. The first rabbit received an aerosol of physiological saline solution (0.9% NaCl), the second one received aerosolized low dose of bromelain 0.01 mg/mL (accumulative dose of 0.025 mg bromelain in 2.5 mL saline), the third rabbit received aerosolized high dose of bromelain 0.1 mg/mL (accumulative dose of 0.25 mg bromelain in 2.5 mL saline), over 11 min inhalation period.

During the experiment, animal's body weight, gut stasis, signs of allergy or bleeding, appearance, body condition, natural behaviour and provoked behaviour were monitored. Observation during the two post-operative days indicated that rabbit's activities were normal, with normal food, drink intake and normal breathing. Rabbits were euthanized 48 h post-treatment using IV Sodium pentobarbitone 100 mg/kg while animals were sedated using isoflurane. Blood samples and internal organs were collected for pathology, with histopathology, quantitative assays for bromelain concentration in plasma and blood coagulation assays being conducted using standard procedures.

### Histopathological Results

Two rabbits were used in the first pilot experiment. A control rabbit (physiological saline solution) and treated one (0.1 mg/mL bromelain). In comparison with the control rabbit, the treated rabbit showed mild cytoplasmic vacuolisation of hepatocytes around central veins. The tracheal epithelium and bronchial epithelium and their structures were intact. Both control and treated rabbits showed pulmonary haemorrhage. No other abnormalities were found in the bromelain-treated lungs.

Three rabbits were used in the second pilot study, control rabbit (physiological saline solution) and two treated rabbits (a low dose of 0.01 mg/mL bromelain and a higher dose of 0.1 mg/mL bromelain). Transverse sections from the gastroesophageal junction from the control and the treated animals showed no injuries. Samples of trachea of the 3 animals were also obtained. Histopathological examination of the trachea tissues as well as section of the bronchi and bronchioles showed intact airway epithelia and structures with no injuries. Subcellular examination of airways cilia showed normal structures.

The amount of drug which entered the systemic circulation via the pulmonary and gastrointestinal routes would be metabolised by the liver or excreted by the kidneys. Hence, liver and kidney samples were taken for histopathological analysis. Transverse sections from the livers of the 3 animals showed no differences between bromelain-treated animals and the control including parenchyma and the portal area. There was no signs of inflammatory effects or blood extravasation. No subcellular differences between liver parenchymal cells of control and treated rabbits. Kidneys of the 3 animals were intact. Investigation of the cortex and medulla of the kidneys showed no histopathological alteration in Bowman's capsules and the tubules including absence of inflammation and blood extravasation. No subcellular changes in kidneys were observed.

Histological examination of the lungs of the three rabbits showed pulmonary haemorrhage, like that observed in control and treated rabbits in the first experiment. This finding is also in accordance with the hematoma observed by post-mortem gross examination of the lungs. This pathological finding is potentially due to the use of isoflurane for anaesthesia during treatment and euthanasia. Histopathological examination of lungs also showed inflammation observations in both control and low-dose bromelain treated rabbits but not in high-dose bromelain treated rabbit. By comparison with control rabbits, inhalation of an accumulative dose of 0.19-0.25 mg Bromelain in physiological saline caused similar effects on lungs (Table 6).

TABLE 6
Summary of histopathological outcome

| Procedure | Treatment | Outcome |
|---|---|---|
| 1 | Control | Pulmonary haemorrhage. |
| | | Mild pneumonitis. |
| | Brom 0.19 | Pulmonary haemorrhage, multifocal to diffuse, mild to moderate. |
| | mg | |
| 2 | Control | Pulmonary haemorrhage, multifocal to diffuse, mild to moderate. |
| | | Mild pneumonitis, granulomatous. |
| | Brom 0.025 | Pulmonary haemorrhage, multifocal to diffuse, mild to moderate. |
| | mg | Mild pneumonitis, mixed inflammatory infiltration. |
| | Brom 0.25 | Pulmonary haemorrhage, multifocal to diffuse, mild to moderate. |
| | mg | |

Lungs are poorly perfused with marked artefactual atelectasis present.

Pulmonary haemorrhage has been observed in all control and treated rabbits. The inventors found that isoflurane can induce pulmonary capillary haemorrhage and that VEGF, which causes capillary permeability/leakage is induced by isoflurane. Isoflurane was inhaled by the rabbits during the treatment as well as induction of euthanasia. Use of isoflurane was therefore avoided in the next rabbit inhalation studies (described below). The marked artefactual atelectasis in the current pilot studies has been attributed to poor perfusion of the lungs with fixative, which was avoided as well in the subsequent rabbit inhalation studies.

### Blood Plasma Results

In the 1st experiment, plasma concentration of bromelain in the treated rabbit was 800 ng/mL 48 h post-treatment. In the 2nd experiment, ELISA assay showed no traces of bromelain in plasma samples of rabbits collected at 4, 6, 24 and 48 h post-inhalation of either 0.025 mg or 0.25 mg accumulative doses of bromelain.

In the 2nd experiment, blood samples were collected 4, 6, 24 and 48 hr post-inhalation. ELISA assay and a standard curve ranging from 0-1200 ng/mL Rabbit factor X (F10) (CUSABIO, catalogue number #CSB-EL007915RB) was used to evaluate factor X in plasma. No significant changes were found at 4, 6, and 24 h post treatments between control and treated rabbits (either Bromelain 0.01 or 0.1 mg/mL). At 48 h post treatments, rabbits were anaesthetized using Isoflurane before euthanasia and blood collection. There was no change in the F10 concentration in plasma of 0.1 mg/mL (total of 0.25 mg) dose treated rabbit. A decrease in Factor X concentration in control and 0.01 mg/mL (total of 0.025 mg)-treated rabbit was observed.

In conclusion, accumulative doses of 0.025, 0.19 or 0.25 mg of Bromelain in 0.9% physiological saline solution were safe when administered by inhalation in rabbits as there is no difference between control and treated rabbits.

### Example 2.2—Tolerability Study of Inhaled BromAc in Rabbit Model

In this second pilot study. 24 SPF rabbits (Flinders University, SA. AU) were used to test the safety of different doses of Bromelain in combination with Acetylcysteine when delivered via inhalation in a rabbit model.

The rabbits were equally divided into 6 treatment groups, each rabbit receiving one of the following treatments with accumulative doses of Bromelain and Acetylcysteine as follows:
Treatment A: 0.9% sterile saline solution (Control) (N=4)
Treatment B: Acetylcysteine 20 mg (N=4)
Treatment C: Bromelain 0.1 mg (N=4)
Treatment D: Bromelain 0.25 mg (N=4)
Treatment E: Bromelain 0.1 mg and Acetylcysteine 20 mg (N=4)
Treatment F: Bromelain 0.25 mg and Acetylcysteine 20 mg (N=4)

The treatment dose of Acetylcysteine alone, Bromelain alone or in combination with Acetylcysteine in 2.5 mL saline was nebulized for inhalation to each rabbit using a nebulizer and snout mask. Pre-treatment, rabbits were sedated using a mixture of Acepromazine 1 mg/kg body weight SC and Medetomidine 0.25 mg/kg body weight SC. All rabbits were administered pre-operative Ventolin inhalation (Salbutamol 800 micrograms/Kg) as a bronchodilator to combat potential bronchoconstriction effects of the drug. At least 10 min period has been kept between Salbutamol administration and treatment.

Post-treatment, animals were monitored every 15 mins in the first hour, then every hour up to the first 6-8 hours post-treatment; then at 12-hour post-treatment; then 3 times the next day (morning, midday, and evening with 4 hours in-between) and then 48 hr post-treatment (before euthanasia). The inhalation treatment was carried out in the morning to allow close monitoring of the animals. During the experiment, animal's body weight, gut stasis, RbtGS scale (attached to ethics protocol), respiratory distress, hypoxia, signs of allergy or bleeding, appearance, body condition, natural behaviour and provoked behaviour were monitored. Blood samples were collected from rabbit's ear veins 4, 6, 24 and 48 h post-treatment.

Two days post-treatment, all treated animals were euthanized. In the first pilot study, pulmonary haemorrhage was observed, which was attributed to the anaesthesia and euthanasia agents used. To minimize these effects, the following procedure was used in the current study. First, rabbits were sedated with the injection of a mixture of Acepromazine 1 mg/kg body weight and Medetomidine 0.25 mg/kg body weight-SC. Then, rabbits were injected with a mixture of pentobarbitone 50 mg/kg and potassium chloride 1 mmol/kg IV. After euthanasia, post-mortem, comparative observations, collection of lungs and other internal organs were made for pathology. Lungs were perfused for using 10% Formalin for 10 minutes before extraction to avoid artefactual atelectasis.

Similar, histopathology, quantitative assays for bromelain concentration in plasma and blood coagulation assays to those described above in Example 2.1 were performed. The results indicated that inhalation of bromelain alone or in addition to acetylcysteine had no effect on the general health of the rabbits as indicated by parameters of general wellbeing, pain, and distress. Post-treatment examinations also indicated that there were no signs of allergy, bleeding, hypoxia, or respiratory distress in all treated rabbits. Post-mortem examination showed no apparent gross differences between lungs of test and control rabbits. The changes in the lungs of these rabbits were quite varied from mild to moderate. Histological assessment of the lungs showed a degree of interstitial pneumonia in all control and treated lungs. In contrast to the pilot study, haemorrhage in the lungs is not seen here, just scanty occasionally intra-alveolar haemorrhages. Histological assessment of the livers, kidneys and hearts did not show morphological differences between control and treated rabbits. Table 7 summarises the pathological findings in the rabbit's tolerability study.

Bromelain or acetylcysteine were not detected in plasma samples of rabbits at 4, 6, 24 and 48 h post-inhalation of accumulative doses of 0.1 or 0.25 mg bromelain alone or in combination with acetylcysteine, as shown by ELISA assay.

According to obtained results, Bromelain in combination with Acetylcysteine (i.e. BromAc) is safe to be delivered by an aerosol into the respiratory tract at the concentrations examined in this model.

TABLE 7
Summary of histopathological outcome of the respiratory airways-Rabbit safety study
(N = 4/group).

| | | Incident |
|---|---|---|
| Treatment | Outcome | (out of 4) |
| Control | Interstitial pneumonia, multifocal, scanty | 1 |
| | Interstitial pneumonia, multifocal, mild granulomatous | 3 |
| | Interstitial inflammatory infiltrates | 1 |
| | Perivascular inflammatory infiltrates | 2 |
| | Scanty intra-alveolar haemorrhages | 1 |
| | Rare vascular thrombi | 1 |
| Ac 20 mg | Broncho-interstitial pneumonia, multifocal, mild, granulomatous | 3 |
| | Broncho-interstitial pneumonia, multifocal, mild | 1 |
| Brom 0.1 mg | Broncho-interstitial pneumonia, multifocal, mild, granulomatous | 1 |
| | Broncho-interstitial pneumonia, multifocal, mild | 2 |
| | Interstitial pneumonia, multifocal, mild, granulomatous | 1 |
| Brom 0.25 mg | No significant abnormalities | 1 |
| | Broncho-interstitial pneumonia, multifocal, mild | 1 |
| | Interstitial pneumonia, multifocal, mild | 2 |
| | Perivascular inflammatory infiltrates | 1 |
| | Scanty Vasculitis | 1 |
| Brom 0.1 | Broncho-interstitial pneumonia, multifocal, mild granulomatous | 1 |
| mg + Ac 20 | Interstitial pneumonia, multifocal, mild | 2 |
| mg | Interstitial mixed inflammatory infiltrates | 2 |
| | Perivascular inflammatory infiltrates | 4 |
| | Scanty intra-alveolar haemorrhage | 1 |
| Brom 0.25 | Broncho-interstitial pneumonia, multifocal, mild | 2 |
| mg + Ac 20 | Broncho-interstitial pneumonia, multifocal, moderate, mixed | 1 |
| mg | Interstitial pneumonia, multifocal, mild granulomatous | 1 |

### Example 2.3—Safety of Inhaled Bromelain and Acetylcysteine in Mice

After arriving in the BRC animal facility, 72 mice were housed for at least one week for acclimatization under the standard protocol for nude mice. The study was divided into 4 categories (I, II, III and IV). Each category contained three groups (N=6 per group). One of these groups was a sham control group (Group A). The other two groups (Group B, C) were treated with Bromelain and N-acetylcysteine combinations. Animals were identified using the tail marking procedure with a permanent marker. Animals of the treatment categories (I, II, III or IV) were euthanized on 5-, 34-, 10- or 39-days post-start of treatment respectively. Marking was checked during monitoring, if fade, the tail was remarked.

Category I: Exposure 3×/day for 5 consecutive days; a total of 15 doses (Exposure at experimental day 1-5, euthanasia at day 5):
Group A. Control (vehicle)
Group B. Brom 0.250 mg/ml+Ac 20 mg/ml
Group C. Brom 0.500 mg/ml+Ac 20 mg/ml

Category II: Exposure 3×/day for 5 consecutive days; a total of 15 doses (exposure at experimental day 1-5, recovery for 28 days (day 6-33), euthanasia at day 34):
Group A. Control (vehicle)
Group B. Brom 0.250 mg/ml+Ac 20 mg/ml
Group C. Brom 0.500 mg/ml+Ac 20 mg/ml

Category III: Exposure 3×/day for 10 consecutive days; a total of 30 doses (exposure at experimental day 1-10, euthanasia at day 10):
Group A. Control (vehicle)
Group B. Brom 0.250 mg/ml+Ac 20 mg/ml
Group C. Brom 0.500 mg/ml+Ac 20 mg/ml

Category IV: Exposure 3×/day for 10 days; a total dose of 30 doses (exposure at experimental day 1-10, recovery for 28 days (day 11-38), euthanasia at day 39):
Group A. Control (vehicle)
Group B. Brom 0.250 mg/ml+Ac 20 mg/ml
Group C. Brom 0.500 mg/ml+Ac 20 mg/ml

One exposure dose of Bromelain in combination with N-acetylcysteine per time was inhaled by each group (N=6). The mice were caged in a standard mice cage and treated using an inhalation chamber. A metal lid was fit on the cage, the lid having outlet holes and an inlet connected to a Philips Respironics InnoSpire Deluxe Compressor Nebuliser. Mice received the inhalation treatments for 10 minutes/dose according to the above schedule. The concentration of the aerosol within the breathing zone of the chamber was continuously monitored using a DustTrak 8520 light scatter device (TSI, St Paul, MN) and maintained in a way that each mouse received continuously the treatments dose over the 10 min period.

The mice were monitored during the treatment procedure. Category I and III animals were monitored for 30 mins post-exposure and then daily up to their euthanasia. Category II and IV animals were monitored for 30 mins post-exposure and then daily up to Day 10. The animals were then monitored 3 times/week (excluding weekends) up to their euthanasia.

Animals were euthanized by exsanguination (cardiac puncture) following an IP injection of sodium pentobarbitone at a dose of 60 mg/kg diluted in 0.9% saline. A diluted pentobarbitone sodium solution 12 mg/mL was prepared (0.184 mL of the 325 mg/mL stock solution and add 0.9% saline up to 5 mL). The mouse dose was 5 mL/kg (i.e IP injection of 0.1 mL for an average 20 g mouse (Less than 1% of body weight). All the blood and tissue specimens obtained were analysed for possible toxicity and results were compared and contrasted in different groups. Evaluation of cytotoxicity has been carried out by histopathology of the lungs, liver, kidney, spleen, pancreas, and intestine.

The analysis techniques used were similar to those described above and are known in the art.

### Category I Results

Observations made post-inhalation treatment indicated that mice activities were normal, with normal food, drink intake and normal breathing. The results also indicate that inhalation of BromAc had no effect on the general health monitoring score of the mice as indicated by parameters of general wellbeing, pain, and distress including appearance, body condition, natural behaviour and provoked behaviour.

Post-treatment examinations also indicated that there were no signs of haemorrhage, hypoxia, or respiratory distress in all treated mice. In addition, there was no significant change in body weight fluctuation through the treatment course between animals in the treatment groups and the control. All treated groups (control and treated) didn't show in loss of body weight other than on experimental day 1. Further, results showed that there is no significant difference in total number of large cells (WBCs) in Bronchoalveolar Lavage fluid between control and BromAc-treated groups in Category I animals.

Upon lung histology examination, there was increase in number of animals with no lung abnormalities in the BromAC 0.5/20 mg/mL treatment group (3 out of 6), compared to the control group (2 out of 6). Whilst all animals in the BromAC 0.25/20 mg/mL treatment group showed abnormalities, the observed types of abnormalities were also seen in the control group. Comparing the total number of abnormalities/groups, there was only extra one abnormality in the BromAC 0.250/20 mg/mL treatment group (6) compared to the control (5). At an increased drug concentration (the BromAC 0.500/20 mg/mL treatment group), there was a decrease in the total number of abnormalities/group (3) compared to control (5).

By comparison with control mice, treatment of mice with BromAc 0.250/20 mg/mL didn't show histological alterations in livers, kidneys, spleen, pancreases, and intestine in drug-treated mice.

### Category II Results:

Observations made post-inhalation treatment indicated that mice activities were normal, with normal food, drink intake and normal breathing. The results also indicate that inhalation of BromAc had no effect on the general health monitoring score of the mice as indicated by parameters of general wellbeing, pain, and distress including appearance, body condition, natural behaviour and provoked behaviour.

Post-treatment examinations also indicated that there were no signs of haemorrhage, hypoxia, or respiratory distress in all treated mice. Post-treatment recovery period (>5 days) showed normal health monitoring score in both control and treated groups. In addition, there was no significant change in body weight fluctuation through the treatment course between animals in the treatment groups and the control. Remarkably, animals treated with BromAC didn't show loss of body weight other than on experimental day 1. Additionally, the results showed that there was no significant difference in total number of large cells (WBCs) in Bronchoalveolar Lavage fluid between control and BromAc-treated groups in Category II animals.

Upon lung histology examination, all animals treated with BromAC 0.250/20 mg/mL (6 out of 6) showed no abnormalities compared to the control (3 out of 6). There was no difference in the number of animals with no lung abnormalities in the BromAC 0.500/20 mg/mL treatment group compared to control (3 out of 6 for both). By comparison of “total number of abnormalities/groups”, there was no abnormalities in BromAC 0.250/20 mg/mL group (0) compared to control (4). In addition, by increasing drug concentration, BromAC 0.500/20 mg/mL group showed decrease in total number of abnormalities/group (2) compared to control (4).

By comparison with control mice, treatment of mice with BromAc 0.500/20 mg/mL didn't show histological alterations in livers, kidneys, spleen, pancreases, and intestine in drug-treated mice.

### Category III Results

Observations made post-inhalation treatment indicated that mice activities were normal, with normal food, drink intake and normal breathing. The results also indicate that inhalation of BromAc had no effect on the general health monitoring score of the mice as indicated by parameters of general wellbeing, pain, and distress including appearance, body condition, natural behaviour and provoked behaviour.

Post-treatment examinations also indicated that there were no signs of haemorrhage, hypoxia, or respiratory distress in all treated mice. In addition, there was no significant change in body weight fluctuation through the treatment course between animals in the treatment groups and the control. All treated groups (control and treated) didn't show in loss of body weight other than on experimental day 1. Further, the results showed that there is no significant difference in total number of large cells (WBCs) in Bronchoalveolar Lavage fluid between control and BromAc-treated groups in Category III animals.

Upon lung histology examination, there was decrease in number of animals with no lung abnormalities in the BromAC 0.250/20 mg/mL treatment group (2 out of 6), compared to the control group (4 out of 6). However, the number of animals with no lung abnormalities in the BromAC 0.50/20 mg/mL treatment group (4 out of 6) was similar to control group. By comparison of “total number of abnormalities/groups”, there was 4 abnormalities in BromAC 0.250/20 mg/mL group (6) compared to control (2). However, by increasing drug concentration, BromAC 0.500/20 mg/mL group showed decrease in total number of abnormalities/group (only 1) compared to control (5)

By comparison with control mice, treatment of mice with BromAc 0.500/20 mg/mL didn't show histological alterations in livers, kidneys, spleen, and pancreases in drug-treated mice.

### Category IV Results:

Observations made post-inhalation treatment indicated that mice activities were normal, with normal food, drink intake and normal breathing. The results also indicate that inhalation of BromAc had no effect on the general health monitoring score of the mice as indicated by parameters of general wellbeing, pain, and distress including appearance, body condition, natural behaviour and provoked behaviour.

Post-treatment examinations also indicated that there were no signs of haemorrhage, hypoxia, or respiratory distress in all treated mice. Post-treatment recovery period (>10 days) showed normal health monitoring score in both control and treated groups. In addition, there was no significant change in body weight fluctuation through the treatment course between animals in the treatment groups and the control. Remarkably, animals treated with BromAc didn't show loss of body weight than experimental Day 1. Additionally, results showed that there is no significant difference in total number of large cells (WBCs) in Bronchoalveolar Lavage fluid between control and BromAc-treated groups in Category IV animals.

Upon lung histology examination, 5 out of 6 animals treated with BromAc 0.250/20 mg/mL group showed “no lung abnormalities”, while control group showed abnormalities in 4 animals out of 6, and there were abnormalities in 3 animals in BromAc 0.500/20 mg/mL group (3 out of 6). By comparison of “total number of abnormalities/groups”, there was only 2 abnormalities in BromAC 0.250/20 mg/mL group compared to 5 abnormalities in control animals and 3 in BromAC 0.500/20 mg/mL group.

By comparison with control mice, treatment of mice with BromAc 0.500/20 mg/mL didn't show histological alterations in livers, kidneys, spleen, and intestine in drug-treated mice.

The pathological findings in the lungs of mice in the inhalation chamber study are shown below in Table 8.

TABLE 8
Pathological findings in the lungs of mice in the inhalation chamber study

| | Treatment | | Incident |
|---|---|---|---|
| Category | mg/mL | Pathological findings | (Out of 6) |
| I | Control | No abnormalities. | 2 |
| | | Mild (1+) alveolar haemorrhages. | 1 |
| | | Mild (1+) multifocal alveolar haemorrhages. | 2 |
| | | Mild (1+) multifocal perivascular haemorrhage and oedema | 1 |
| | | with rare erythrophagocytosis by alveolar macrophages. | |
| | | Mild (1+) perivascular lymphocytic infiltrate. | 1 |
| | | Multifocal atelectasis which is interpreted as an artefact of | 3 |
| | | poor perfusion of the lung tissue with formalin post mortally. | |
| | BromAc | Mild (1+) multifocal alveolar haemorrhages. | 5 |
| | 0.25/20 | Mild (1+) multifocal perivascular and alveolar macrophage | 1 |
| | | accumulation. | |
| | | Multifocal atelectasis which is interpreted as an artefact of | 5 |
| | | poor perfusion of the lung tissue with formalin post mortally. | |
| | BromAc | No abnormalities. | 3 |
| | 0.5/20 | Mild (1+) multifocal alveolar haemorrhages. | 1 |
| | | Mild (1+) multifocal perivascular haemorrhages. | 2 |
| | | Multifocal atelectasis which is interpreted as an artefact of | 3 |
| | | poor perfusion of the lung tissue with formalin post mortally. | |
| II | Control | No abnormalities. | 3 |
| | | Mild (1+) perivascular lymphocytic infiltrate. | 3 |
| | | Mild (1+) multifocal alveolar and intra-bronchiolar | 1 |
| | | haemorrhages. | |
| | | Multifocal atelectasis which is interpreted as an artefact of | 2 |
| | | poor perfusion of the lung tissue with formalin post mortally. | |
| | BromAc | No abnormalities. | 6 |
| | 0.25/20 | | |
| | BromAc | No abnormalities. | 3 |
| | 0.5/20 | Mild (1+) increase in the numbers of alveolar macrophages. | 1 |
| | | Mild (1+) multifocal alveolar and intra-bronchiolar | 1 |
| | | haemorrhages. | |
| | | Multifocal atelectasis which is interpreted as an artefact of | 1 |
| | | poor perfusion of the lung tissue with formalin post mortally. | |
| III | Control | No abnormalities. | 5 |
| | | Diffuse vascular congestion | 1 |
| | | Mild (1+) multifocal alveolar haemorrhages. | 1 |
| | BromAc | No abnormalities. | 2 |
| | 0.25/20 | Scant (1+) multifocal alveolar haemorrhages. | 1 |
| | | Mild (1+) multifocal alveolar haemorrhages. | 1 |
| | | A focus of alveolar mineralisation- incidental background | 1 |
| | | finding | |
| | | Mild (1+) multifocal perivascular lymphocytic infiltrate. | 1 |
| | BromAc | No abnormalities. | 5 |
| | 0.5/20 | Mild (1+) perivascular lymphocytic infiltrate. | 1 |
| IV | Control | No abnormalities. | 2 |
| | | Mild (1+) hypercellularity of alveolar walls with lymphocyte | 3 |
| | | infiltration | |
| | | Mild (1+) multifocal alveolar haemorrhages. | 2 |
| | BromAc | No abnormalities. | 6 |
| | 0.25/20 | Mild (1+) hypercellularity of alveolar walls with lymphocyte | 1 |
| | | infiltration | |
| | | Mild (1+) multifocal alveolar haemorrhages. | 1 |
| | BromAc | No abnormalities. | 3 |
| | 0.5/20 | Mild (1+) hypercellularity of alveolar walls with lymphocyte | 2 |
| | | infiltration | |
| | | Mild (1+) multifocal perivascular lymphocyte, plasma cell | 1 |
| | | and macrophage infiltration. | |

According to obtained results, it is safe for multiple administrations of BromAc to be delivered by an aerosol into the respiratory tract. Such treatment regimens are likely to be needed for treating chronic respiratory diseases and conditions.

### Example 3—Cytokine and Chemokine Inhibition in Tracheal Aspirate Mucus of COVID-19 Patients

These experiments describe the mucolytic and anti-inflammatory effect of BromAc in tracheal aspirate samples obtained from critically ill COVID-19 patients requiring invasive mechanical ventilation.

Tracheal aspirate (TA) samples (2-10 ml) were collected during the early morning routine of COVID-19 patients, aging from 18-80 years-old, under mechanic artificial ventilation at the intensive care unit of three hospitals in Brazil. All patients included in the study tested positive for SARS-COV-2 by RT-PCR targeting for E gene. Only secretive productive patients were included in the study. Samples were aspirated into sterile tracheal secretion collectors and immediately processed in a biosecurity level 3 laboratory. Tracheal aspirate samples were treated with the combination of Bromelain (125 and 250 μg) and NAC 2% (20 mg/ml) (BromAc) at different concentrations of Bromelain and incubated at 37° C. After incubation, flow through assays, rheometric measurements and cytokine storm assessment were performed. All responsible family members signed the written informed consent on behalf of patients and agreed to participate in the investigation. This study followed the principles of Helsinki declaration as well as the resolution #466/2012 from the Brazilian Ministry of Health for research involving humans.

All data followed parametric distribution and therefore were analysed by ANOVA one way followed by post-hoc Dunnet's multiple comparisons test amongst groups. All the analysis were carried out using GraphPad Prism, version 8.0, (San Diego, CA, USA). Significant statistical differences were considered if p value was less or equal 0.05.

### Example 3.1—Determination of BromAc's Mucolytic Efficacy in Tracheal Aspirate Samples from COVID-19 Patients

### Flow Through Assay

For assessing the impact of BromAc and its mucolytic effect in the viscosity of tracheal aspirate (TA) samples from COVID-19 patients. TA specimens were treated with 125 to 250 μg of Bromelain and 2% NAC (BromAc) using a mucosal atomization device (MAD nasal) (Teleflex) and incubated at 37° C. in 6 well plates. After incubation and visual assessment of TA specimens treated with BromAc, time of mucolysis was assessed. Then, treated TA samples were passed through cell strainers (70 μm) placed into 50 mL falcon polypropylene tubes. Samples were pipetted into cell strainers and volume flow through was collected and measured. Mincing and repeated pipetting was strictly avoided in order to measure flow through accurately. Volume flow through was harvested, aliquoted and stored at −80° C.

### Rheometric Measurements

Rheometry was also utilized for assessing the viscosity and shear stress promoted by BromAc treatment. The evaluation of the rheological profile of the tracheal aspirate samples was carried out in a Brookfield rotational viscometer, model DV III (Ametek Brookfield, Middleborough, USA). Analysis was performed using three sample groups: control (no treatment), NAC (treated with n-acetylcysteine). BromAc 125 (treated with Bromelain at 125 μg and NAC 2%) and BromAc 250 (treated with Bromelain at 250 μg and NAC 2%). After preparation and addition of the active principles as performed previously, all samples were homogenized for 1 minute and placed in a water bath at 37° C. for 30 minutes. Subsequently, 1 mL of each sample was placed in the sampling cup of the rotational viscometer and the reading was obtained using the CP52 spindle. The equipment was controlled using Rheocalc V3.3 software (Ametek Brookfield, Middleborough, USA) programmed to vary the spindle speed from 0.01 to 250 RPM, with an evaluation of 30 points between these values and equilibrium time 30 seconds at each new speed. Viscosity (cP), torque (%) and shear stress (D/cm²) data were collected, and the results were presented in terms of shear rate (sec-1).

Determination of BromAc's Efficacy in Tracheal Aspirate Samples from COVID-19 Patients

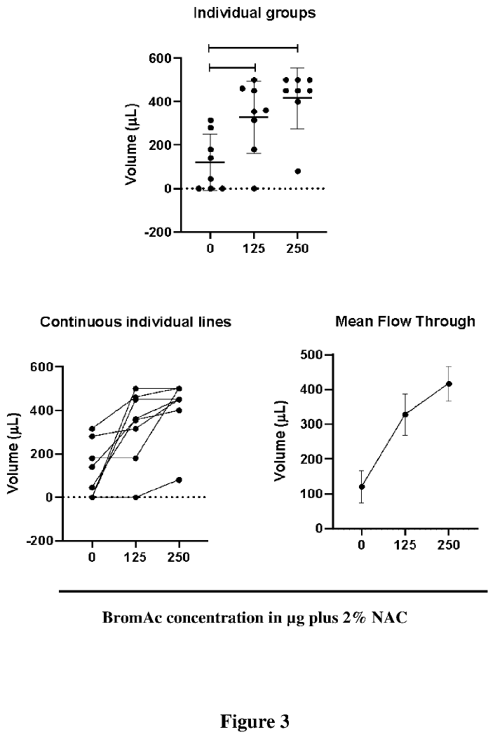

In order to determine whether BromAc treatment would exert a mucolytic effect on tracheal aspirate samples in a dose-dependent manner, samples were treated with NAC alone at 2% as well as BromAc 125 mcg and 250 mcg (with 2% NAC). Tracheal aspirate samples were treated for 30 minutes under the conditions described in material and methods. Tracheal aspirate samples from eight (8) COVID-19 patients were tested. Results demonstrate robust mucolytic effect of BromAc in a dose-dependent manner as shown in FIG. 3. Flow through recovery was 68% for samples treated with BromAc 125 mcg and over 80% for samples treated with BromAc 250 mcg.

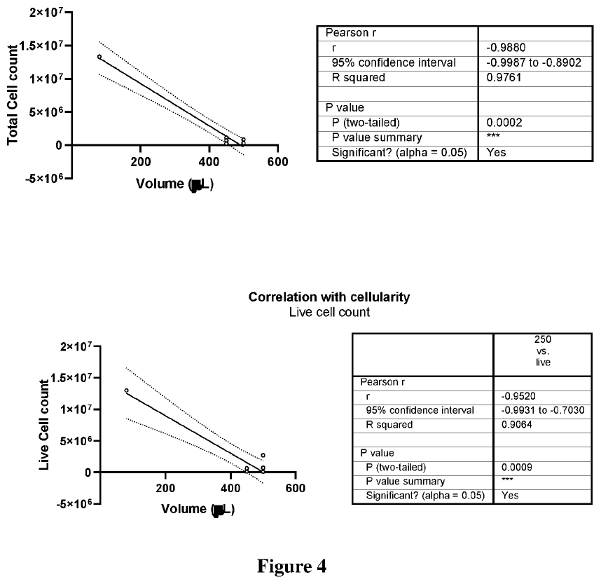

Cellularity of tracheal aspirate samples from COVID-19 patients was then assessed after treatment with BromAc 250 mcg. Results are shown in FIG. 4. The data demonstrates that cellularity correlates inversely with the mucolytic effect of BromAc (p=0.0002, Pearson correlation R square=−0.9880). In addition, this correlation was seen also with the live cell compartment assessed in all samples BromAc (p=0.0009, Pearson correlation R square=−0.9520).

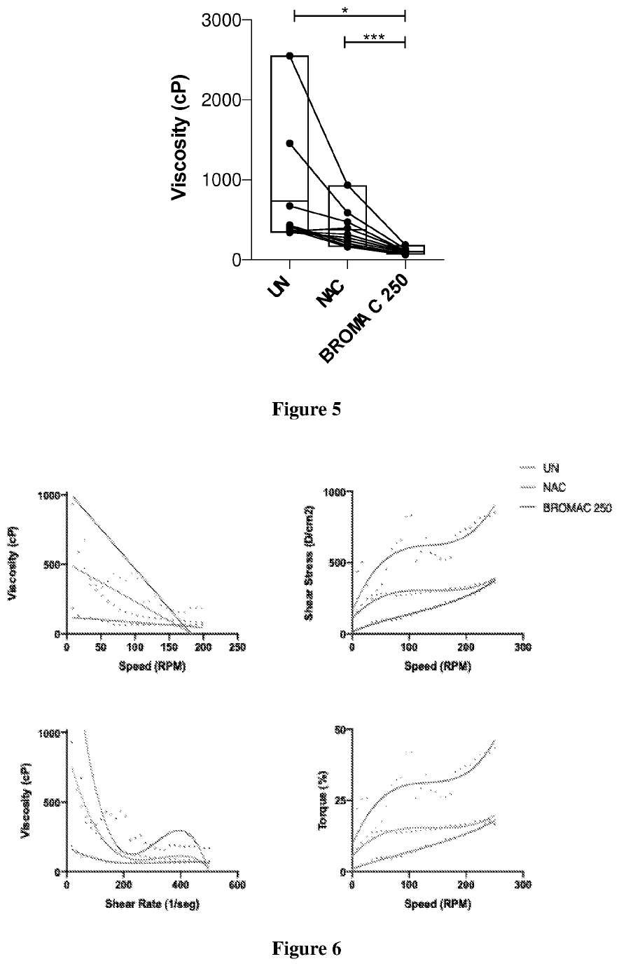

As another method of measuring the impact of BromAc treatment on the viscosity of tracheal aspirates from COVID-19 patients, rheometric measures were performed to assess viscosity, shear rate, shear stress and torque on samples upon BromAc treatment. FIG. 5 shows the results of 10 replicated measures of one tracheal aspirate sample from a COVID-19 patient.

The Viscosity, shear stress, shear rate as well as torque were assessed on tracheal aspirate samples from a COVID-19 patient along the speed (of the spindle). The results are shown in FIG. 6. Rheometric measures display conspicuous and statistically significant differences amongst the untreated versus NAC and BROMAC-treated samples. The results demonstrate significant impact of BromAc 250 mcg in tracheal aspirate samples from COVID-19 patients when compared to both NAC and untreated samples.

### Example 3.2—Anti-Inflammatory Activity of BromAc in Tracheal Aspirate Samples from Critically Ill COVID-19 Patients

Assessment of Immunological Inflammatory Mediators in Tracheal Aspirate Samples from COVID-19 Patients

In order to verify the effect of BromAc in the inflammatory mediators present in tracheal aspirate (TA) samples from COVID-19 patients. TA specimens were treated with 125 to 250 μg of Bromelain and 2% NAC (BromAc) using the MAD device as described previously. Treated and untreated TA specimens were incubated at 37° C. in 6 well plates for 1 hour. After that, TA specimen aliquots were initially cleared by centrifugation at 800×g for 10 min, at room temperature and TA supernatants were transferred to fresh 2 mL microtubes. Samples were diluted 1:10 and incubated with magnetic beads covered with monoclonal antibodies specific to several immunological mediators, such as: chemokines (CXCL8, CCL11, CCL3, CCL4, CCL2, and CXCL10), inflammatory cytokines (IL-1b, IL-6, TNF-α, IL-12, IFN-g, IL-15 and IL-17), regulatory cytokines (IL-1Ra, IL-9. IL-10) and growth factors (FGF-basic, PDGF, VEGF, G-CSF. GM-CSF, IL-2 and IL-7). Experiments were carried out according to the manufacturer's instructions using the Procarta Human Cytokine 27-plex Assay, (Invitrogen, CA, USA). The immunological mediators were measured in TA samples and the concentrations of each sample was determined according to standard curves run for each molecule tested using a fifth parameter logistic fit analysis. The results were expressed as pg/mL for all mediators tested.

Anti-Inflammatory Activity of BromAc in Tracheal Aspirate Samples from Critically Ill COVID-19 Patients

In order to determine BromAc's effect on the immune components present in the tracheal aspirate samples from COVID-19 patients, the detection of chemokines, cytokines and growth factors was performed after treatment with BromAc. This experiment was performed in similar conditions as the previous curve experiment in which samples were treated with NAC alone at 2% as well as BromAc 125 mcg and 250 mcg. Tracheal aspirate samples were treated for 60 minutes under the same conditions as the first experiment.

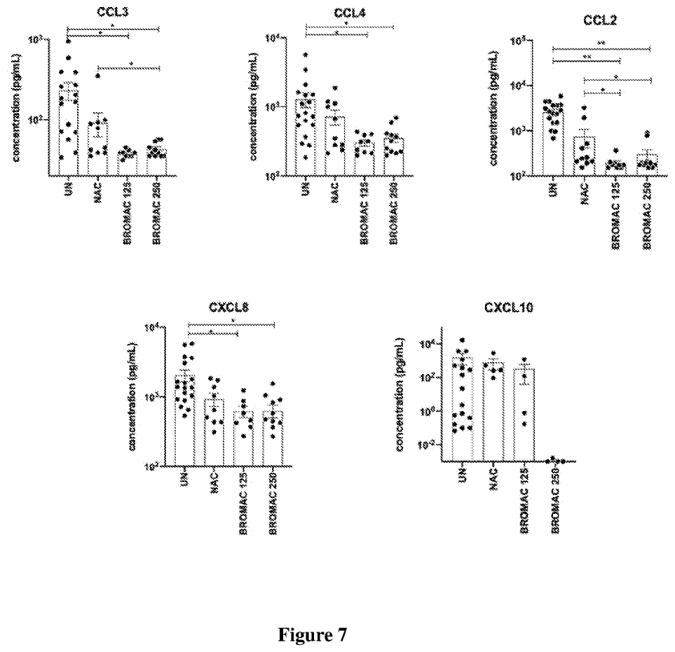

Results show that BromAc treatment generally and massively abrogated the chemokines (FIG. 7). Of note, BromAc decreased the expression of chemokines CCL2, CCL3, CCL4 and CXCL8, which are associated to proinflammatory neutrophils and macrophage recruitment to the lungs.

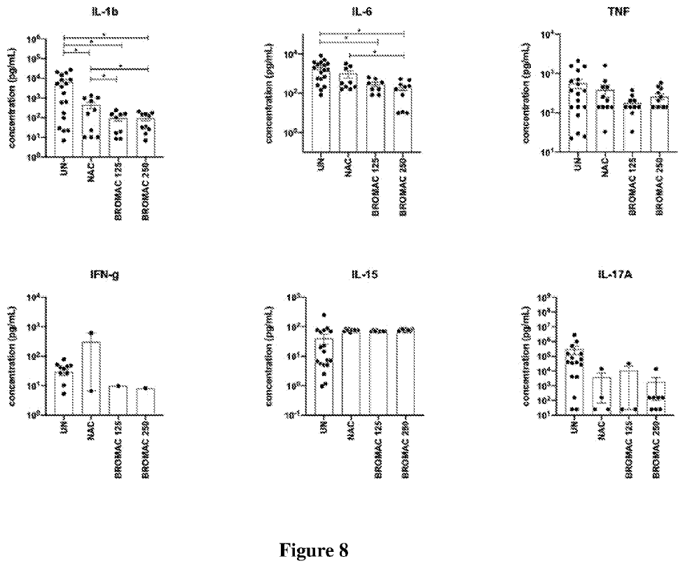

Furthermore, the cytokine storm was generally altered by BromAc treatment, showing lower levels of IL-6, a major inflammatory player on COVID-19 alongside IL-15, IFN-g and IL-17A (FIG. 8). Alongside decreased levels of proinflammatory cytokines IL-1b in association with IL-6 were observed after treatment with BromAc, indicating possible modulation of inflammasome-associated pathway. IL-1b and TNF were decreased in samples treated with BromAc.

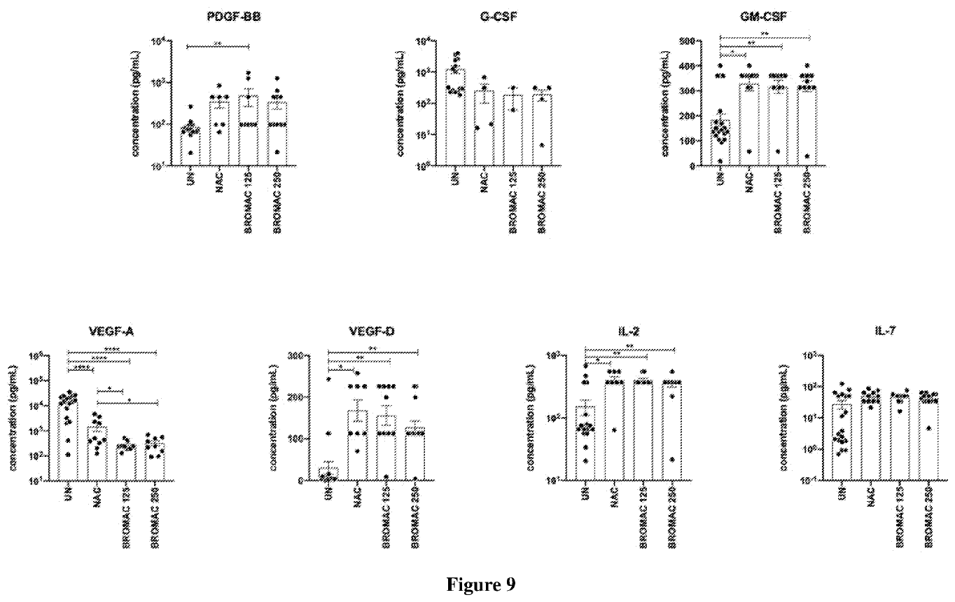

The analysis of the ex vivo activity of BromAc on growth factors in the tracheal aspirate samples from COVID-19 patients indicated decreased levels of and vascular endothelial growth factor. VEGF-A, in opposition to other growth factors such as VEGF-D. IL-2, PDGF and GM-CSF which were increased after treatment with BromAc (FIG. 9). Increased PDGF, a growth factor associated with platelet recruitment, should be better studied, but it could be associated to the degradation of platelets, which leads to the release of this growth factor. The same hypothesis could be drawn by other growth factors that could be membrane bound and detach from cells after BromAc treatment.

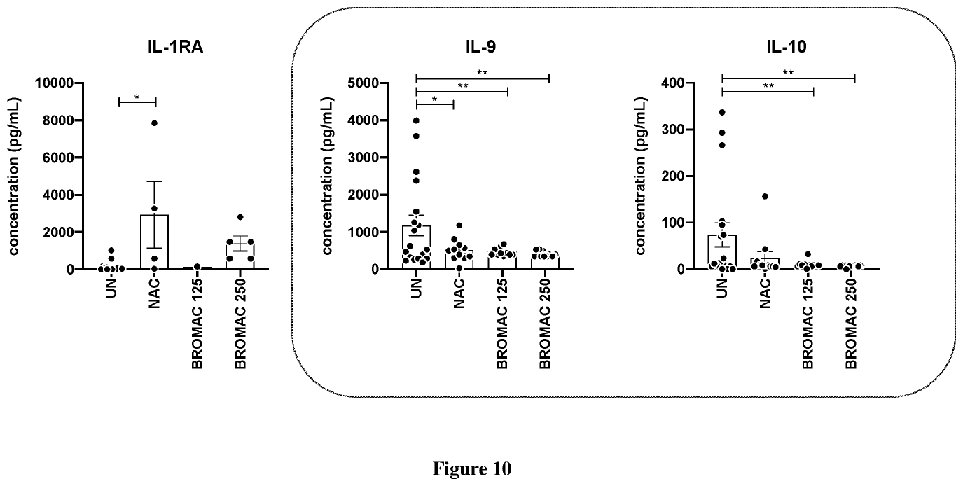

Regarding the regulatory cytokines, the results show major abrogation of these molecules (FIG. 10). IL-1RA levels were not significantly altered by BromAc treatment as compared to NAC, however, decreased levels of IL-9 and IL-10 were observed when BromAc groups were compared to untreated tracheal aspirate samples. For IL-9, the same effect was observed in NAC-treated group, however, for IL-10, the significant decrease in the levels of this cytokine was solely observed in the BromAc groups. Excessive regulation may be associated or may lead to decreased responsiveness, anergy and diminished effector responses against the virus, therefore, these results may indicate that BromAc could contribute to maintaining full effector responses, such as Type 1 immune responses against SARS-COV-2 during the viral phase.

### Conclusions

The results described above demonstrate that BromAc displays robust mucolytic effect in tracheal aspirate samples from COVID-19 patients in a dose dependent manner. In addition, BromAc showed anti-inflammatory activity, reducing the action of cytokine storm in tracheal aspirate samples from patients with COVID-19. BromAc also resulted in a reduction in chemokines such as MIP-1alpha, CXCL8, MIP-1b, MCP-1 and IP-10, compared to NAC alone and control. In addition, there was action on regulatory cytokines such as IL-5, IL-10, IL-13 IL-1RA and total reduction for IL-9. Even at a concentration of 250 mcg Brom (plus 20 mg NAC), it acted on IL-6, one of the main pro-inflammatory cytokines and on growth factors, demonstrating a reduction in G-CSF and VEGF-D at concentrations of 125 mcg and 250 mcg.

These results indicate robust mucolytic and anti-inflammatory effect of BromAc in tracheal aspirates from critically ill COVID-19 patients, indicating its potential as a therapeutic strategy to COVID-19 as well as for therapeutic applications in other conditions where cytokine storms play a detrimental role or in which inhibiting cytokine activity may be beneficial.

### Example 4—Effect of Nebulised NAC, Bromelain and BromAc on the Rheological Properties of Artificial and Simulated Sputa

Respiratory diseases such as cystic fibrosis can be difficult to treat owing to viscous secretions in the airways that evade mucocilliary clearance. The effect of nebulised NAC, bromelain and BromAc on gelatinous airway representative sputa models was therefore investigated. The two sputa models used in this study are artificial sputa (AS) and simulated sputa (SS), which was a mucinous secretion from pseudomyxoma peritonei (PMP) patients that has been treated as described below to represent thick airway sputa.

### Materials and Reagents

For preparing artificial sputa the following materials were purchased from Sigma Aldrich, Sydney, Australia: Porcine mucin, Salmon sperm DNA, potassium chloride, sodium chloride, TRISMA Base. TPTZ (2,4,6-Tripyridyl-s-triazine), Ferrous chloride. PMP mucin of soft grade was obtained from a clinical sample that had been assessed for its softness index. Pipettes (0.5 ml), capillary tubes, endotracheal tubes size 9.0, Nebulizer equipment (PHILIPS Respironics InnoSpire Essence Nebulizer Compressor, flow rate 7 L per minute, 10 psi).

### Artificial Sputa Preparation

Artificial sputum was prepared following protocol as detailed by Kitchner et al (Kirchner, S., et al., Use of artificial sputum medium to test antibiotic efficacy against Pseudomonas aeruginosa in conditions more relevant to the cystic fibrosis lung. Journal of visualized experiments: JOVE, 2012 (64)). Briefly 250 mg of porcine mucin, 200 mg of sperm DNA, 295 mg of diethylene triamine pentaacetic acid (DPTA), 25 mg sodium chloride, 110 mg potassium chloride, 140 mg Tris base were mixed in a volume of 45 ml of distilled water and pH adjusted to 7.0 using Trisbase. The volume of the solution was then adjusted to 50 ml. Further dilutions were then carried out to adjust viscosity as desired.

### Preparation of PMP Mucin as a Model of Sputa

Six grams of soft PMP mucin was homogenised using a shredder with phosphate buffer saline (PBS) (3.0 ml) with sonification and vortexed until a homogenous mixture was formed, with incubation at 37° C. to remove air bubbles and after which viscosity was adjusted (further dilutions with PBS). The pH was adjusted to 7.0 using either 1.0 M NaOH or 0.1 N Hydrochloric acid.

### Measurement of Dynamic Viscosity

Dynamic viscosity (Y) of sputum was measured using the capillary tube method as outlined in Santander, J. and G. Castellano, Determination of the kinematic viscosity by the liquid rise in a capillary tube. Revista Brasileira de Ensino de Física, 2013. 35.

### Measurement of Pipette Flow Time

Using a 0.5 ml glass pipette fixed at an angle of 60°, 0.5 ml of sample at 25° C. (ambient room temperature) was sucked up the pipette and the time taken to empty 0.3 ml of the sample was timed in sec. Pipette flow time (€) was calculated as follows:

€ (ml/sec)=0.3 ml/Time to empty 0.3 ml (sec)

Treatment with Aerosol Acetylcysteine

Artificial sputa (AS) were prepared as detailed above, and 1.5 ml of artificial sputum was carefully emptied into an endotracheal tube (in triplicates). A volume of 6.0 ml of NAC 10 mg/ml in PBS (pH.7.0) in triplicate was aerosolised using a nebulizer and passed over the sputum samples in the endotracheal tubes kept at 37° C. in a water bath. Similarly, the experiments were repeated using NAC 20 mg/ml in PBS (pH.7.0). The controls only received PBS (pH 7.0). The aerosol delivery time to empty 6.0 ml for each treatment was 25 min.

The dynamic viscosity of the sputa was measured before and after using the capillary tube method, whilst the pipette flow time was also measured as described earlier using a 0.5 ml glass pipette. Samples were equilibrated to 25° C. before measurement

A similar investigation as above was carried out to determine a comparative dynamic viscosity and pipette flow time for PMP mucin simulated sputa (SS).

Treatment with Nebulised Bromelain (Artificial Sputa, Simulated Sputa)

A similar treatment as in the experiment described above for NAC was carried out using Bromelain (125 and 250 μg/ml) in PBS (pH.7.0) along with control using PBS (pH.7.0). Both dynamic viscosity and pipette flow time was measured as outlined earlier for both artificial and PMP simulated sputa models.

Treatment with Aerosol BromAc (Bromelain+NAC) (Artificial Sputa, Simulated Sputa)

Using a combination of NAC (20 mg/ml) with either 125 or 250 μg/ml Bromelain in PBS (pH.7.0), a similar investigation was carried out as outlined earlier and measurements were made for both artificial and simulated sputa models.

### Measurement of Bromelain in Aerosolised Sputum Samples

Suitable dilutions such as 1/5 and 1/10 of nebulised samples in PBS were carried out with subsequent filtration (0.44 um). To 250 μl of sample was added 250 μl of 1% azocaesin solution (prepared in distilled water). The samples were agitated at room temperature (25° C.)/1 hrs, after which 1.5 ml of 1% trichloroacetic acid was added, vortexed and centrifuged at 2500 RPM. To 150 μl of supernatant in a microwell is added an equal quantity of 0.5 M Sodium hydroxide solution and the OD at 410 nm was read using a spectrometer.

A standard curve for Bromelain was generated following a similar procedure with Bromelain dilutions ranging from 200 μg/ml that was serially diluted.

### Measurement of Acetylcysteine in Sputum Samples

Suitable dilution such as 1/5 and 1/10 of aerosolised samples and filtration, as above was carried out. 10 mM solutions of TPTZ were prepared in distilled water (dH2O). Stock solution of NAC 10 mg/ml was prepared in PBS and pH adjusted to 7.0. Stock solution (10 mM) of Fe (III) was prepared in dH₂O. The pH of all the reagents was adjusted to 7.0.

To 125 μl of TPTZ was added 125 μl of Fe (III) solution vortex mixed and then followed by the addition of 100 μl of test solution. It is then vortexed and placed on a gentle shaker for 1 hour at ambient room temperature (25° C.) until full colour (blue) develops. To this is then added 2.0 ml of dH2O, vortex mixed, and the OD is measured using aliquots of 200 μl in triplicates into a 96 well plate. Blanks only contained 100 μl of dH₂O. The OD at 593 nm was read using UV spectrometer (Shimadzu). Suitable dilutions of Acetylcysteine ranging from 200 μg/ml down was prepared for the generation of standard curve.

### Calculation of D Values (%) for Both the Dynamic Viscosity (γ) and Pipette Flow (€)

D=(TREATED)−(UNTREATED/(UNTREATED)×100.

### Example 4.1—Effect of NAC Aerosol on the Rheological Properties (Dynamic Viscosity and Laminar Flow) of Artificial Sputum and PMP Simulated Sputum

Table 9 shows the effect of aerosolised N-Acetylcysteine on both artificial and simulated sputa using two parametric measurements such as dynamic viscosity and pipette flow time. NAC: N-Acetylcysteine. ⬇=decrease; ⬆=increase.

TABLE 9
Dynamic Viscosity and Pipette flow speed

| | ARTIFICIAL SPUTUM (AS) | | | SIMULATED SPUTUM (SS) | | |
|---|---|---|---|---|---|---|
| Treatment | Before | After | D (%) | Before | After | D (%) |

DYNAMIC VISCOSITY (γ) MEASUREMENTS (cSt)

| | | | | | | |
|---|---|---|---|---|---|---|
| Control | 1.382 ± 0.013 | 1.373 ± 0.001 | 0.65 ↓ | 1.748 ± 0.006 | 1.713 ± 0.004 | 2 ↓ |
| NAC 10 mg/ml | | 1.300 ± 0.002 | 6.0 ↓ | | 1.467 ± 0.011 | 16 ↓ |
| NAC 20 mg/ml | | 1.247 ± 0.041 | 9.8 ↓ | | 1.453 ± 0.055 | 17 ↓ |

PIPETTE FLOW SPEED (€) (ml/sec)

| | | | | | | |
|---|---|---|---|---|---|---|
| Control | 0.005 ± 0.001 | 0.006 ± 0.001 | 20 ↑ | 0.0137 ± 0.005 | 0.0153 ± 0.001 | 12 ↑ |
| NAC 10 mg/ml | | 0.0064 ± 0.003 | 28 ↑ | | 0.0183 ± 0.001 | 34 ↑ |
| NAC 20 mg/ml | | 0.007 ± 0.001 | 40 ↑ | | 0.02 ± 0.002 | 46 ↑ |

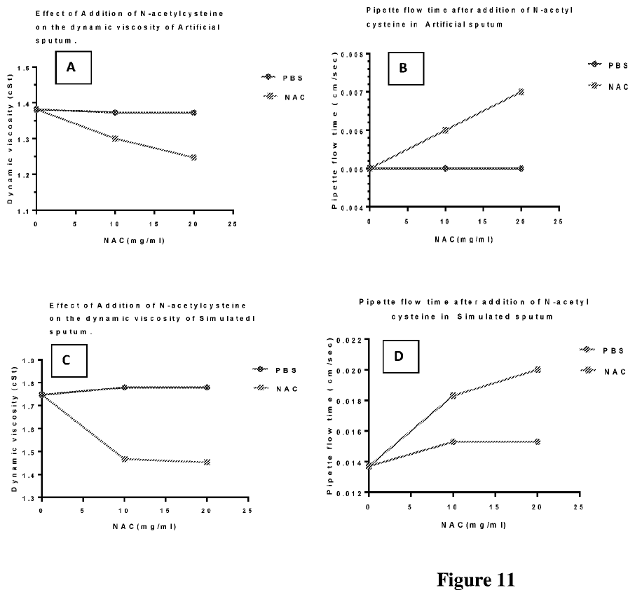

As can be seen from Table 9 and FIGS. 11A-D, treatment with PBS had very minor effect on dynamic viscosity (γ) of artificial sputum (AS), however, NAC at 10 and 20 mg/ml showed a marked decrease in γ, 6.0 and 9.8%, respectively. The pipette flow speed (€) indicated that treatment with PBS affected it by 20% indicating that hydration may play a substantial role on this parameter. Further, treatment with NAC 10 and 20 mg/ml also showed a corresponding increase in pipette flow (28 and 40%, respectively).

In the case of simulated sputa (SS), PBS treatment affected γ slightly (2%) while having almost an equal effect (16 and 17%) with treatment of NAC at the two concentration (10 and 20 mg/ml). Further there was also a corresponding increase in € values. Thus, both the sputa models were affected by aerosol treatment with NAC.

FIG. 11A indicates that in artificial sputa (AS), with the addition of N-Acetylcysteine, there is a noticeable concentration dependent reduction of dynamic viscosity. B) shows that in AS there is a linear increase in pipette sputum flow with the addition of increasing amount of NAC. C) indicates that there is a considerable drop in dynamic viscosity of simulated sputum (SS) with the addition of NAC 10 and 20 mg/ml, as compared to control. D) shows the increase in pipette sputum flow after the addition of increasing amounts of NAC in SS model.

### Example 4.2—Effect of Bromelain Aerosol on the Rheological Properties (Dynamic Viscosity and Laminar Flow) of Artificial Sputum and PMP Simulated Sputum

Table 10 shows the effect of aerosolised Bromelain (BR) on both artificial (AS) and simulated sputa (SS) using two parametric measurements, dynamic viscosity (γ) and pipette flow time (€). ⬇=decrease; ⬆=increase

TABLE 10
Dynamic viscosity (cSt) and Pipette flow speed

| | ARTIFICIAL SPUTUM (AS) | | | SIMULATED SPUTUM (SS) | | |
|---|---|---|---|---|---|---|
| Treatment | Before | After | D (%) | Before | After | D (%) |

DYNAMIC VISCOSITY(γ) MEASUREMENTS (cSt)

| | | | | | | |
|---|---|---|---|---|---|---|
| Control | 1.382 ± 0.013 | 1.373 ± 0.001 | 0.65 ↓ | 1.748 ± 0.006 | 1.713 ± 0.004 | 2 ↓ |
| BR 125 μg/ml | | 1.012 ± 0.022 | 27 ↓ | | 1.612 ± 0.004 | 8.0 ↓ |
| BR 250 μg/ml | | 0.870 ± 0.003 | 36 ↓ | | 1.600 ± 0.009 | 8.5 ↓ |

PIPETTE FLOW SPEED (€) (ml/sec)

| | | | | | | |
|---|---|---|---|---|---|---|
| Control | 0.005 ± 0.001 | 0.006 ± 0.001 | 20 ↑ | 0.0137 ± 0.005 | 0.0153 ± 0.001 | 12 ↑ |
| BR 125 μg/ml | | 0.0218 ± 0.003 | 336 ↑ | | 0.046 ± 0.001 | 243 ↑ |
| BR 250 μg/ml | | 0.0290 ± 0.001 | 480 ↑ | | 0.073 ± 0.004 | 443 ↑ |

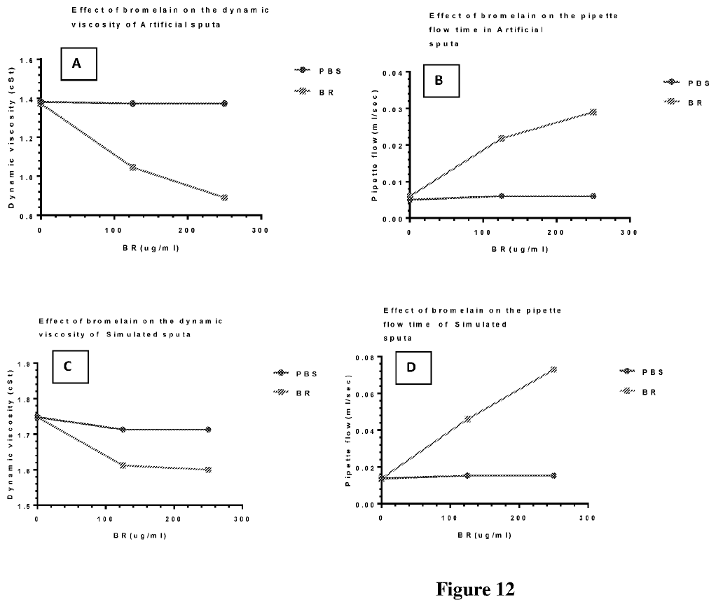

As can be seen from Table 10 and FIGS. 12A-D, the dynamic viscosity (γ) of both the sputa AS and SS were slightly affected by aerosol PBS, 0.65 and 2.0% respectively, however treatment with Bromelain at 125 and 250 μg/ml indicated a noticeable drop in (γ) for both the sputa models, with much higher drops for AS. The effect at the two concentrations of Bromelain in SS model was almost the same. When the pipette flow speed (€) was examined, aerosol PBS showed an effect on both the sputa models 20 and 12% in AS and SS, respectively, however this effect was very high when treated with Bromelain in both the models.

FIG. 12A) shows that in artificial sputum (AS), with the addition of Bromelain (BR) there is a considerable reduction of viscosity in comparison to control (PBS). B) shows that the pipette flow in AS is considerably increased as compared to control (PBS) when treated with Bromelain. C) shows that in simulated sputa (SS), the addition of Bromelain shows a reduction in viscosity in comparison to control (PBS). D) shows that in SS, the pipette flow is highly increased compared to control (PBS), after treatment with Bromelain.

### Example 4.3—Effect of BromAc Aerosol on the Rheological Properties (Dynamic Viscosity and Laminar Flow) of Artificial Sputum and PMP Simulated Sputum

Table 11 shows the effect of aerosolised BromAc® (Bromelain (BR) 125 or 250 μg/ml+NAC 20 mg/ml) on both artificial (AS) and simulated sputa (SS) using two parametric measurements such as dynamic viscosity (γ) and pipette flow time (€). ⬇=decrease; ⬆=increase

TABLE 11
Dynamic viscosity (cSt) and Pipette flow speed

| | ARTIFICIAL SPUTUM (AS) | | | SIMULATED SPUTUM (SS) | | |
|---|---|---|---|---|---|---|
| Treatment | Before | After | D (%) | Before | After | D (%) |

DYNAMIC VISCOSITY(γ) MEASUREMENTS (cSt)

| | | | | | | |
|---|---|---|---|---|---|---|
| Control | 1.382 ± 0.013 | 1.373 ± 0.001 | 0.65 ↓ | 1.748 ± 0.006 | 1.713 ± 0.004 | 2 ↓ |
| BR 125 μg/ml + | | 0.95 ± 0.001 | 31 ↓ | | 1.416 ± 0.033 | 19 ↓ |
| NAC 20 mg/ml | | | | | | |
| BR 250 μg/ml + | | 0.800 ± 0.002 | 42 ↓ | | 1.282 ± 0.027 | 27 ↓ |
| NAC 20 mg/ml | | | | | | |

PIPETTE FLOW SPEED (€) (ml/sec)

| | | | | | | |
|---|---|---|---|---|---|---|
| Control | 0.005 ± 0.001 | 0.006 ± 0.001 | 20 ↑ | 0.0137 ± 0.005 | 0.0153 ± 0.001 | 12 ↑ |
| BR 125 μg/ml + | | 0.033 ± 0.002 | 556 ↑ | | 0.061 ± 0.002 | 343 ↑ |
| NAC 20 mg/ml | | | | | | |
| BR 250 μg/ml + | | 0.035 ± 0.001 | 600 ↑ | | 0.114 ± 0.002 | 733 ↑ |
| NAC 20 mg/ml | | | | | | |

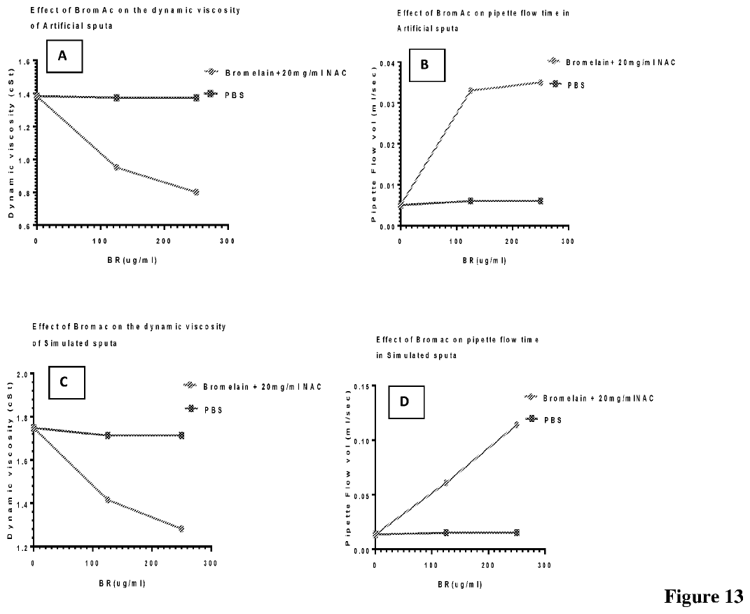

As can be seen from Table 11 and FIGS. 13A-D, treatment with Bromelain at the two concentrations (125 and 250 μg/ml) with NAC 20 mg/ml had a noticeable effect on (γ) with sufficient decrease in both the sputa models. This was further indicated in (€) values with very high increase in both the models.

FIG. 13A) shows that in artificial sputa (AS) there is a noticeable drop in dynamic viscosity after treatment with BromAc (Bromelain 125 and 250 μg/ml+NAC 20 mg/ml). B) shows that in AS the flow rate increase with the addition of BromAc. C) shows that in simulated sputa (SS) there is a considerable drop in dynamic viscosity with the addition of 125 or 250 μg/ml Bromelain+NAC 20 mg/ml. D) shows that in SS, there is almost a linear increase in flow rate (pipette emptying time) with the addition of increasing amount of Bromelain and 20 mg/ml NAC.

### Example 4.4—Accumulation of Bromelain and Acetylcysteine in Artificial Sputum and PMP Simulated Sputum

Table 12, shows the concentration of Bromelain (BR) and Acetylcysteine (NAC) in the two sputa models (AS and SS) after passage of nebulised solution over the sputa with either just Bromelain, NAC, or their combination over 25 minutes.

TABLE 12
Concentration of Bromelain (BR) and Acetylcysteine (NAC)

| | ARTIFICIAL | | | |
|---|---|---|---|---|
| | SPUTA (AS) | | SIMULATED SPUTA (SS) | |
| Reagent | BR (μg/ml) | NAC (mg/ml) | BR (μg/ml) | NAC (mg/ml) |
| BR 125 μg/ml | 30.58 ± 0.211 | | 57.91 ± 0.562 | |
| BR 250 μg/ml | 58.63 ± 0.89 | | 61.64 ± 0.88 | |
| NAC 10 mg/ml | | 2.75 ± 0.143 | | 1.85 ± 0.310 |
| NAC 20 mg/ml | | 2.90 ± 0.210 | | 2.367 ± 0.133 |
| BR 125 μg/ml + | 41.23 ± 1.212 | 2.41 ± 0.122 | 77.56 ± 3.12 | 1.62 ± 0.093 |
| NAC 20 mg/ml | | | | |
| BR 250 μg/ml + | 79.15 ± 2.22 | 2.22 ± 0.184 | 88.21 ± 4.11 | 2.16 ± 0.132 |
| NAC 20 mg/ml | | | | |

As can be seen from Table 12 and FIGS. 14A-C, bromelain at 125 μg/ml as an individual agent showed a concentration of 30.58 μg/ml in the AS and with 250 μg/ml it was 58.63 μg/ml, indicating that doubling the concentration almost doubled the sequestered Bromelain. However, in the SS, there was very little difference between the two Bromelain concentrations (57.91 vs 61.64 μg/ml). NAC as individual agent showed only a small difference in concentration between 10 and 20 mg/ml in AS whilst slightly larger difference in SS models. When Bromelain was delivered in NAC 20 mg/ml, Bromelain concentration in the 125 μg/ml was half of that found in the 250 μg/ml Bromelain in the AS model whilst in the SS model, there was only a 11% difference. Although NAC 20 mg/ml was delivered with either 125 or 250 μg/ml Bromelain, NAC analysis indicated that in the AS model, the difference was small with a slightly larger difference in the SS model.

FIG. 14A) shows that the concentration of Bromelain in the artificial sputa (AS) increases almost 2-fold with exposure to two-fold increase in concentration of aerosolised Bromelain (125 vs 250 μg/ml). However, the difference was relatively small for the SS, simulated sputa 58 vs 62 μg/ml. B) shows that the difference between the low (10 mg/ml) and the high (20 mg/ml) NAC in the artificial sputa (AS) was relatively small (difference of 0.15 mg/ml) when exposed to aerosolised NAC. However, in the simulated sputa (SS), the difference was relatively larger (1.85 vs 2.37 mg/ml), about 28% increase. C) shows comparative levels of Bromelain in artificial and simulated sputa before and after addition of 20 mg/ml NAC to the aerosolised solution. The concentration of Bromelain is relatively higher in both the sputa models, in the presence of NAC 20 mg/ml.

The D values of both dynamic viscosity (γ) and flow speed (€) between artificial and simulated sputa were also compared. It has been shown above that a small changes in viscosity of the mucinous samples can affect large flow rate based on pipette emptying time, which may have a bearing on ciliary clearance.

Although, with the addition of NAC 20 mg/ml to Bromelain 125 and 250 μg/ml in artificial sputum, the decrease in viscosity was only 4-6% compared to Bromelain alone (from 31-42% to 27-36%, respectively), there was tremendous increase in flow speed (€) indicating that small decrease in viscosity may affect clearance of the fluid by ciliary motion. The high BromAc concentration group indicates almost twice the reduction of pipette emptying time compared to the low BromAc group. This does not correlate with their respective dynamic viscosity (γ) that may indicate that high glycosylated mucin may affect the viscosity to a significant level. In simulated sputa, with the addition of NAC 20 mg/ml to Bromelain 125 and 250 μg/ml, the decrease in dynamic viscosity was 19 and 27% compared to 8 and 8.2%, respectively. Further, a similar trend in flow speed (€) was seen in both sputa.

Table 13 shows a comparison of Dynamic viscosity (Y) and Flow speed (€) between the two sputa models when treated with Bromelain 125 or 250 g/ml and in combination with NAC 20 mg/ml.

TABLE 13
Dynamic viscosity (γ) and Flow speed (€)
D value (%)

| | Artificial Sputa (AS) | | Simulated Sputa (SS) | |
|---|---|---|---|---|
| | Dynamic | Flow Speed | Dynamic | Flow Speed |
| Treatment | Viscosity (γ) | (€) | Viscosity (γ) | (€) |
| 125 μg/ml BR | 27 | 336 | 8.0 | 243 |
| 250 μg/ml BR | 36 | 480 | 8.2 | 443 |
| 125 μg/ml BR + | 31 | 556 | 19 | 343 |
| NAC 20 mg/ml | | | | |
| 250 μg/ml BR + | 42 | 600 | 27 | 733 |
| NAC 20 mg/ml | | | | |

Table 13 shows a comparison of D values (for both the dynamic viscosity (Y) and flow speed (€) between artificial sputa (AA) and simulated sputa (SS) treated with Bromelain only and with the addition of NAC 20 mg/ml. With the addition of NAC 20 mg/ml, there is a slight increase in (γ) in AS whilst the difference was much larger in SS model. Further, a similar trend in (€) was seen in both the sputa.

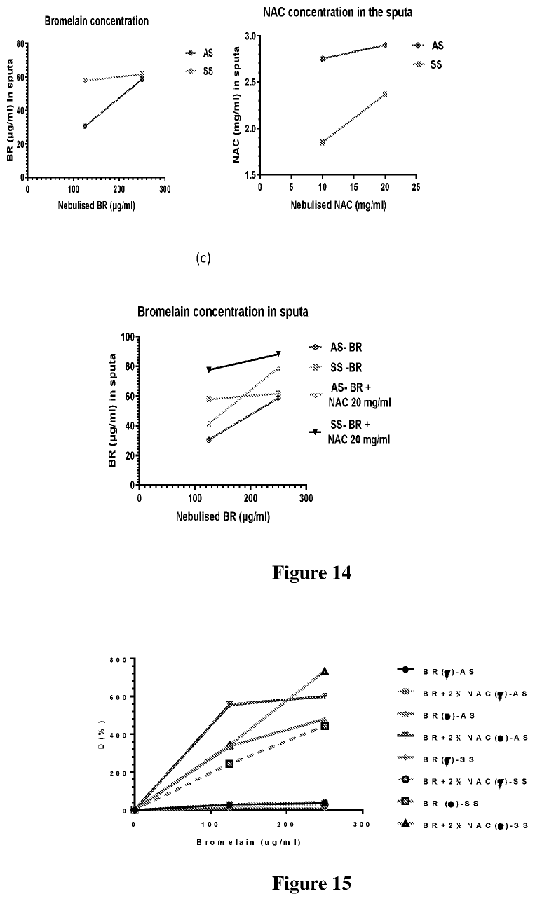

FIG. 15 shows the relative differences in enhancement D (%) of the two parameters measured such as dynamic viscosity (γ) and flow speed € in the artificial sputum (AS) and simulated sputum (SS), when treated with either Bromelain (BR) 125 or 250 μg/ml alone or in combination with NAC 20 mg/ml (BromAc). Viscosity changes in the different groups are amplified in flow speed showing the effect of viscosity on the latter. Treatment with BromAc has a much higher effect on flow speed compared to treatment with bromelain alone.

### Discussion of the Results of Example 4

Since clearance of airway secretion is mainly dependent on its rheological parameters, the dynamic viscosity (γ) and the flow speed (€) of the AS and SS sputa (formulated to represent thick and static sputa) were measured before and after treatment with Acetylcysteine, Bromelain and BromAc. The differences for both γ and € (pre-treated as opposed to treated) were calculated as a percentage denoted by D. Additionally, the sequestration of Bromelain and NAC in the sputa before and after aerosol delivery was investigated to assess whether there was a correlation between the concentrations of the agents within the sputa with the changes in rheological properties.

Treatment of artificial sputa (AS) with aerosolised PBS indicated a minute drop in γ (0.65%) that may be mainly due to hydration, whilst in both the NAC 10 and 20 mg/ml, the reduction was by 6.0 and 10%, respectively. NAC is a well-known antioxidant and hence the reduction of disulfide linkages found in the protein and mucin components may be responsible for this change in viscosity. In comparison, simulated sputa (SS) in PBS showed a 2.0% drop in γ with substantial effect in NAC 10 and 20 mg/ml (γ=16 and 17%, respectively). This difference between both the sputa may be due to their differences in composition and variability of the constituents.

The effect on flow speed € was substantial (20% increase) with PBS treatment, indicating that hydration alone may have considerable effect on the viscoelastic property of sputum in AS. Further, at NAC 10 and 20 mg/ml, the effect was 28 and 40% respectively. In, comparison, the SS displayed a substantial increase in flow speed € with only PBS (12%) whilst both the NAC 10 and 20 mg/ml had a much higher impact (34 and 46% increase). The differences between the two sputa models in flow speed may be mainly due to their differences in composition. Most likely, the percentage of disulfide bonds in the SS model may be much higher compared to AS model and hence their reduction by NAC shows a considerable effect on this parameter.

Treatment with Bromelain showed a marked drop in γ in both the treatment groups (0.125 and 250 μg/ml) with a drop of 27 and 36%, respectively in AS model with substantial effect on € (336 and 480%, respectively) indicating the impact of dynamic viscosity on the flow speed of sputa and ciliary clearance. In the case of SS model again there was drop in γ of 8.0 and 8.2% for the two concentrations of Bromelain (125 and 250 μg/ml). This similarity in γ may be due to the high glycosylation and high mucin present with the sputa. However, the effect of Bromelain on γ amplified the effect on the € values as indicated (243 and 443%). Hence, Bromelain seems to effect both the parameters monitored showing that its hydrolytic properties on proteins and glycoproteins affect the rheological properties of both the sputa models. The variation in efficacy between the two models may be attributed to their differences in composition.

In comparison to NAC, Bromelain has a much greater effect on the two rheological parameters monitored in AS, whilst the impact although less on γ, had much higher impact on € values for the SS model, indicating that the enzymic reactions on these models' sputa shows a much higher activity with greater depolymerisation effect that affected the parameters monitored. Of notice, in the SS model, the effect on γ values were higher in the NAC treated compared to Bromelain which may indicate the high disulfide content in the sample sputa.

When the sputa were treated with BromAc (Bromelain 125 and 250 μg/ml+NAC 20 mg/ml), γ was considerably affected as shown by the percentage differences between untreated and treated sputa in AS for both the 125 and 250 μg/ml Bromelain with values of 36 and 42%. Further this effect was amplified in the € parameter with corresponding 556 and 600%. In the case of SS, the γ parameter between 125 and 250 μg/ml Bromelain showed 19 and 27%, with a substantial difference in € values, (343 and 733%, respectively). Hence, the effect on dynamic viscosity was really amplified in € with considerable rise because of treatment with Bromelain plus NAC 20 mg/ml. These variations between the two models may be mainly due to their variability in composition.

The concentration of Bromelain in the sputa after treatment indicated that, with Bromelain alone, AS showed double the concentration when treated with 250 μg/ml compared to 125 μg/ml Bromelain (58.63 vs 30.58 μg/ml, respectively) with some correlation to their observed effect on γ. On the contrary, the concentration of Bromelain in SS was almost similar in both the Bromelain groups (57.91 vs. 61.64 μg/ml), with correlation with their activity, indicating that Bromelain may have accumulated to perhaps saturation in the models over 25 min aerosol delivery. The differences between the two sputa may also be related to their heterogenous composition. In the case of NAC, the concentration measured in the AS sputa seems to correlate with their activity as measured by dynamic viscosity, and likewise for the SS model. A similar correlation was seen with €.

Finally, the analysis for Bromelain in the BromAc treatments indicated that there was a corresponding double the concentration in the 250 μg/ml Bromelain as opposed to 125 μg/ml Bromelain (79 vs 41 μg/ml Bromelain) in the AS model with correlation to their activity. This was not the case with SS model, which showed only about 11 μg/ml less in the 125 μg/ml group (78 vs 88 μg/ml), indicating that near saturation of Bromelain may have taken place. The NAC sequestered was almost similar in both the high and low Bromelain group, since NAC 20 mg/ml was delivered in both the groups in the AS model. However, there was a difference in the SS model with the 125 μg/ml Bromelain having less NAC concentration (1.62 vs 2.16 mg/ml). These fluctuations may again be partly due to differences in composition.

Dynamic viscosity (γ) and flow speed (€) on a comparable basis indicates that that both the sputa models were affected. The γ in SS was affected less by Bromelain as compared to AS and this may be due to their differences in composition, the former having a higher protein and glycosidic linkages compared to the latter. However, when treated in combination with NAC 20 mg/ml as compared to Bromelain alone, the effect on γ was higher in SS indicating that NAC may be playing a crucial role in reduction of disulfide bridges in the sample affecting the rheology of the sputum. On the other hand, when examining, €, both the sputa were well affected. There is a marked difference in € between Bromelain and that in combination with NAC indicating the importance of NAC in depolymerising the sputa. Importantly, these results further emphasise the high impact of these agents (Bromelain, NAC, BromAc®) on γ and € such that any slight increase of the former magnifies the latter. This finding is important particularly in developing formulas for improving the flow and clearance of sputum from the lungs.

The observed rheological effects in these two model sputa, formulated to represent thick and static sputa, enable a reasonable prediction that aerosol delivery of Bromelain and BromAc will have considerable effect on patient sputum from respiratory diseases such as CF. Certainly, the significantly better performance of these two agents compared with NAC, itself an approved agent for clinical use in relieving chest congestion, shows promise of an enhanced therapeutic effect in treating respiratory diseases.

### Example 5—Rheological Effects on Artificial Mucin Treated with BromAc, DNase and their Combination

The experiments described above have shown that aerosolised BromAc is an efficient mucolytic for cystic fibrosis sputa using two model sputa (artificial and simulated sputa). The inventors also note that DNase is currently marketed (under the brand name Pulmozyme) for the treatment of cystic fibrosis sputa, since sputum is often abundant in double stranded DNA (dsDNA), which increases its viscosity. The experiments described below investigate whether the addition of DNase enhances the mucolytic properties of BromAc in artificial sputa specially formulated to represent cystic fibrosis sputa.

Artificial sputa was prepared following methodology with ingredients as in earlier experiments. To 1.5 ml of artificial sputa in a vial was added 0.5 ml of reagent containing the required bromelain, NAC, DNase and their combinations. Control vials included 0.5 ml of PBS only. The mixture was vortex mixed and incubated in a shaker water bath at 37 deg C. At the end of 1 hour, the samples were retrieved, vortex mixed and equilibrated to ambient room temperature before measuring dynamic viscosity and pipette flow.

Dynamic viscosity was determined using the capillary tube method as in earlier experiments, whilst the pipette flow was evaluated using a 0.5 ml pipette inclined at 60 deg angle. The pipette flow was calculated as shown below.

Pipette flow (€)=0.45 ml sputa/time to empty (sec).

Table 14 shows the sputum flow speed after treatment with Bromelain, NAC, DNase and in combination

TABLE 14
Sputum flow speed (ml/sec) (€)

| | (AGENT) | (plus ------------------- DNASE (ug/ml) ---------------) | | | | | |
|---|---|---|---|---|---|---|---|
| Cont(PBS) | BR(250 ug/ml) | +2.0 | +4.0 | +6.0 | +8.0 | +10. | +20 |
| 0.0436 ± | 0.054 ± | 0.057 ± | 0.075 ± | 0.08 ± | 0.09 ± | 0.1125 ± | 0.150 ± |
| 0.002 | 0.003 | 0.002 | 0.004 | 0.001 | 0.004 | 0.012 | 0.016 |
| Cont(PBS) | NAC(20 mg/ml) | +2.0 | +4.0 | +6.0 | +8.0 | +10. | +20 |
| 0.0436 ± | 0.05 ± | 0.075 ± | 0.09 ± | 0.098 ± | 0.1167 ± | 0.130 ± | 0.143 ± |
| 0.002 | 0.002 | 0.003 | 0.002 | 0.003 | 0.024 | 0.004 | 0.001 |
| Cont(PBS) | — | +2.0 | +4.0 | +6.0 | +8.0 | +10. | +20 |
| 0.0436 ± | | 0.0627 ± | 0.075 ± | 0.082 ± | 0.09 ± | 0.1044 ± | 0.1125 ± |
| 0.002 | | 0.001 | 0.011 | 0.001 | 0.005 | 0.012 | 0.001 |
| | BR (250 ug/ml) + | | | | | | |
| Cont(PBS) | NAC(20 mg/ml | +2.0 | +4.0 | +6.0 | +8.0 | +10. | +20 |
| 0.0436 ± | 0.064 ± | 0.0913 ± | 0.101 ± | 0.1098 ± | 0.124 ± | 0.1350 ± | 0.226 ± |
| 0.002 | 0.04 | 0.001 | 0.01 | 0.002 | 0.003 | 0.032 | 0.001 |

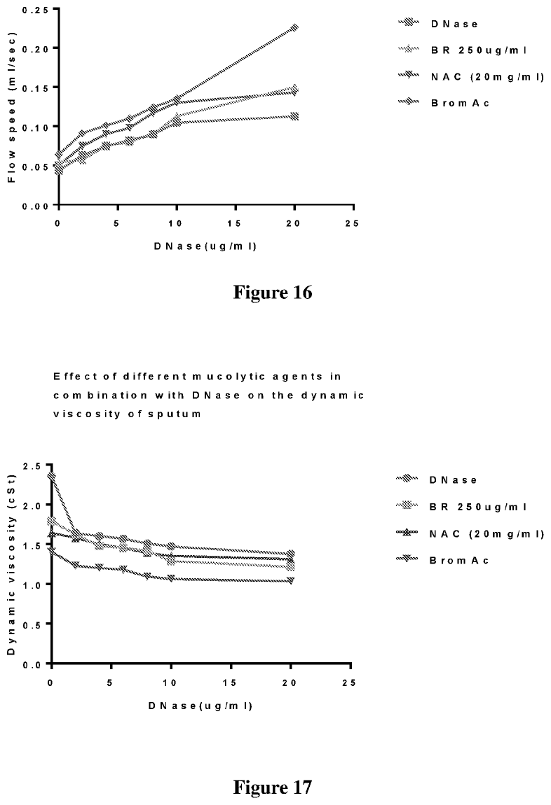

FIG. 16 shows that the pipette flow speed for DNase, as a single agent was almost equivalent to Bromelain (250 μg/ml)+DNase) up to 10 μg/ml, after which further addition of DNase alone did not increase the speed of sputum flow. However, in combination with Bromelain (250 μg/ml), the pipette flow speed increased further. This indicates that initial hydrolysis and change in pipette flow may be mainly due to disruption of phosphodiester bonds found in the dsDNA, after which further hydrolysis of protein (by bromelain) may be the main reason for increase in pipette flow.

In the case of NAC, the addition of DNase increased the flow speed substantially to 10 μg/ml DNase after which there was minimal increase in flow speed. In this reaction, both the reduction of disulfide bonds and hydrolysis of phosphodiester bonds may be contributing to the increase in pipette flow, the flattening of the flow rate is due to exhaustion of substrate. The highest flow speed observed was in the group with DNase+BromAc (bromelain 250 μg/ml+NAC 20 mg/ml), that increased almost in linear fashion all the way to 20 μg/ml DNASE addition. This indicates that the combined action of reducing agent (NAC), along with bromelain (proteolytic) and DNase (phosphodiester hydrolysis) may have contributed together to this enhancement almost linear fashion with the addition of DNase.

Table 15 shows the alteration in sputum dynamic viscosity, after treatment with Bromelain, NAC, DNase and in combination

TABLE 15
Dynamic viscosity (cSt)(¥)

| | (AGENT) | (plus -------------------- DNASE (ug/ml) -------------------------) | | | | | |
|---|---|---|---|---|---|---|---|
| Cont(PBS) | BR(25 ug/ml) | +2.0 | +4.0 | +6.0 | +8.0 | +10. | +20 |
| 2.36 ± | 1.79 ± | 1.623 ± | 1.479 ± | 1.457 ± | 1.416 ± | 1.286 ± | 1.215 ± |
| 0.129 | 0.004 | 0.024 | 0.001 | 0.041 | 0.023 | 0.02 | 0.012 |
| Cont(PBS) | NAC(20 mg/ml) | +2.0 | +4.0 | +6.0 | +8.0 | +10. | +20 |
| 2.36 ± | 1.640 ± | 1.583 ± | 1.502 ± | 1.450 ± | 1.393 ± | 1.354 ± | 1.311 ± |
| 0.129 | 0.021 | 0.003 | 0.01 | 0.033 | 0.013 | 0.01 | 0.001 |
| Cont(PBS) | — | +2.0 | +4.0 | +6.0 | +8.0 | +10. | +20 |
| 2.36 ± | | 1.64 ± | 1.60 ± | 1.57 ± | 1.514 ± | 1.472 ± | 1.379 ± |
| 0.129 | | 0.005 | 0.04 | 0.046 | 0.011 | 0.01 | 0.01 |
| | BR(250 ug/ml) + | | | | | | |
| Cont(PBS) | NAC(20 ug/ml) | +2.0 | +4.0 | +6.0 | +8.0 | +10. | +20 |
| 2.36 ± | 1.406 ± | 1.23 ± | 1.21 ± | 1.185 ± | 1.093 ± | 1.063 ± | 1.035 ± |
| 0.129 | 0.001 | 0.0032 | 0.0021 | 0.033 | 0.014 | 0.003 | 0.01 |

FIG. 17 shows that the effect of DNase as a single agent on dynamic viscosity was less compared to all the other treatment groups since it only hydrolyses the phosphodiester bonds found in the DNA. Bromelain and NAC performed almost equally well with the addition of DNase. However, BromAc with the addition of DNase performed better than all the other groups in reducing the dynamic viscosity, indicating that the addition of DNase may improve the performance of BromAc in solubilising and improving the clearance of airway sputum.

### Discussion

DNase on its own at concentrations ranging from 2-20 μg showed moderate effect on both the rheological parameters monitored compared to combinations with bromelain (250 μg/ml), NAC (20 mg/ml) and BromAc. BromAc+DNase showed higher activity in affecting both the rheological parameters as compared to other treatment groups, suggesting that such a combination might enable the lowering of BromAc concentrations with similar effect on rheological parameters.

The mucolytic enhancement effect of DNase is likely to be dependent on the quantity of double stranded DNA (dsDNA) present within the sputum. DNase is an enzyme with specific properties as a hydrolysing agent (targeting phosphodiester bonds), whilst bromelain has a number of enzymes with variable substrate preference. The combination of agents which have similar therapeutic effect, achieved via different mechanisms shows great potential.

### Example 6—Ventilated Sheep Lung Model

In this study, the inventors used a simulated model of respiratory distress by plugging an endotracheal tube connected to sheep lungs with mucinous sputa. Using clinical ventilators, therapeutic doses of agents such as saline, bromelain, NAC and BromAc were delivered.

10 ml of mucin from pseudomyxoma peritonei (PMP) patients was thoroughly sheared in phosphate buffer saline to simulate a thick/viscous and sticky sputa of purulent cystic fibrosis or of a COVID-19 patient. The mucin from these patients contains MUC1, MUC5AC, cellular debris, double stranded RNA (dsRNA) from degrading cells, lipids and other cellular materials that resemble the sputa of cystic fibrosis after being homogenised and adjusted for viscosity comparable to respiratory mucus plugging.

The lungs (including the trachea) of a freshly slaughtered sheep were donated by Western Sydney Meat Works, and were refrigerated at 4° C. for use within 24-48 hours. Mucin was treated with fresh 10.0 ml of either saline, NAC, bromelain or BromAc twice over 60 minutes (interval of 30 minutes), using the method described below. Each procedure (125 μg/ml or 250 μg/ml bromelain) treatment were conducted in triplicates. Treatment time (time to aerosolise the solution) was 18 minutes, follow up time was 12 minutes and the total treatment time for each sample of mucin was 60 minutes (18+12×2 mins).

A Drager Evita XL ventilator was used in circuit with a size 9.0 endotracheal tube, Aerogen mesh nebuliser and Fisher & Paykel 850 humidifier. The sheep lungs were connected via the trachea to the end of the endotracheal tube. The ventilator was set to continuous mechanical ventilation with the following parameters: Tidal Volume (VT)—0.500 L; Time Inspiratory—2.0; Frequency (f)—8.0 bpm; Pmax—35 cmH₂O; Positive End Expiratory Pressure (PEEP)—0 cmH₂0; Flow—21 l/min. The temperature was maintained at 37° C. Once stable ventilation was established (baseline), mucin was administered into the endotracheal tube to simulate mucus obstruction. The ventilator parameters were recorded as obstruction baseline. BromAc was filled into the in-circuit nebuliser and commenced. Ventilation parameters were recorded every 5 minutes

### Results

Compliance (a measure of how well the lungs respond to ventilation, with higher values being better) monitored over 60 minutes showed an initial increase at 18 minutes that remained almost constant for BromAc. Minute volume (MV) monitored over 60 minutes indicated that there was a slight drop at 18, 30 and 48 minutes for saline treatment, however there was a substantial rise in MV for the other three agents (BromAc. NAC and bromelain), starting at 18 minutes with increases at all the other time monitored. The order of increase with the highest is BromAc followed by the combination of NAC and bromelain, indicating that BromAc had the greatest effect in ventilator parameters at clinically relevant times.

The effect of two concentrations of BromAc (125 and 250 μg/ml) was then assessed. Compliance measurement indicates that BromAc (250 μg/ml bromelain+20.0 mg/ml NAC) showed a sharp increase in compliance at 48 minutes and increased much further at 60 minutes monitored. The compliance readings were much lower for BromAc (125 mg/ml+10 mg/ml NAC) as compared to the higher BromAc concentration. Similarly, minute volume (MV) indicated that there was a sharp rise at 18 minutes that continued to rise at all other time period monitored for BromAc at high concentration.

These data are encouraging for indicating BromAc's use in muco-obstructive disease.

### Example 7—Safety of Nebulised Delivery of BromAc in Healthy Volunteers

12 healthy volunteers were recruited to phase I study on the safety of BromAc as nebulised therapy. The volunteers were otherwise well and remained in the outpatient setting. BromAc, along with 0.9% normal saline, was delivered into the respiratory tract via nebulised aerosol delivery (inhalation) through a mask. All participants were assessed for symptoms and side effects.

The aim of the study was to determine whether nebulised delivery of BromAc at three concentration levels (see Table 16) was safe. The participant received nebulised BromAc at the allocated dose level for a total of 3 days. Symptoms and side effects were reported by the participant, the study nurse or investigator, with specific questions to assess the primary aim of this study.

Dose escalation occurred at the completion of each treatment level (day 3). Study participants received the drug at the levels indicated in a sequential dose design. The first four participants were commenced on dose level 1 (starting dose) and escalation occurred following assessment of safety.

TABLE 16
Dosages used in Phase I clinical trial
Phase I-Healthy Volunteer Dose
Escalation Schedule

| | Dose (5 ml with 0.9% normal saline) | |
|---|---|---|
| | | Ac |
| | Brom | (mg/ |
| Dose Level | (ug/ml) | ml) |
| Level 1 (starting dose) | 100 | 20 |
| Level 2 | 150 | 20 |
| Level 3 | 200 | 20 |

Each participant had their baseline clinical observations taken, including blood pressure, heart rate, respiratory rate, SpO₂ and temperature, chest auscultation for cardiorespiratory assessment, as well as nasal and mouth mucosal assessment. The drug product was then nebulised at a flow rate of 7-8 L/min via a Hudson mask.

Clinical observations were taken every 10 minutes during nebulisation, and then at 30 minutes, 1 hour, 2 hours and 3 hours. Cardiorespiratory auscultation and nasal and mouth mucosal assessment was completed with a Total Nasal Symptom Score (TNSS) being determined at the end of nebulisation and prior to discharge at 3 hours. The participant was asked to monitor for any symptoms or side effects during and after the nebulisation and to complete a symptoms and side effects (patient reported outcomes) form at 30 minutes and at 3 hours. The participant was discharged from clinic after 3 hours monitoring and review by physician investigator. Follow up occurred each treatment day (days 1-3), then on day 4, 5, 10 and 14.

The preliminary results of these clinical trials are described below.

All 12 patients recruited completed the treatment and follow up requirements in this study. There were no observed side effects between dose escalation levels that might be attributed to the increasing dosing level. Four participants (33%) reported a runny nose after nebulisation, for an average of 15 minutes. One participant reported a single episode of diarrhoea on day 1 of treatment. One participant developed viral infection over the course of the study with baseline bloods showing commencement of an inflammatory process, but there was no impact to the patient's overall outcome or treatment observed. One participant reported chest tightness and wheezing following exertional outdoor activity on day 5. The participant attributed these symptoms to cold air and reported that they resolved on day 6.

Additional follow up measurements, including blood parameters, vital signs, mucosal and cardiopulmonary assessments in all participants were comparable to baseline.

In conclusion, the administration of BromAc at the concentrations examined was shown to be safe in healthy volunteers up to 14 days post inhalation based on patient reported outcomes, symptom and side effect assessment, vital signs, blood testing and other clinical observations. Safety will be further examined in patients with respiratory diseases in phase 1b and 2 studies.

The experiments described above evidence that combinations in accordance with the present invention (provided in the form of BromAc) can be administered into the airways of rabbit, mice and human models without adverse effects. The data also shows that nebulised BromAc has a therapeutic effect, at least in that it degrades the types of mucins expected to be found in respiratory diseases. The inventors believe that these data support the therapeutic indications described herein.

As described herein, the present invention provides compositions and methods for treating diseases and conditions such as respiratory diseases, as well as for inhibiting cytokine activity and reducing inflammatory response, in which methods a glycoprotein affecting protease, such as bromelain, is administered by inhalation. Embodiments of the present invention provide a number of advantages over existing therapies, some of which are described above.

It will be understood to persons skilled in the art of the invention that many modifications may be made without departing from the spirit and scope of the invention. All such modifications are intended to fall within the scope of the following claims.

It will be also understood that while the preceding description refers to specific forms of the microspheres, pharmaceutical compositions and methods of treatment, such detail is provided for illustrative purposes only and is not intended to limit the scope of the present invention in any way.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A method for treating a respiratory disease in a patient, the method comprising administering via inhalation a therapeutically effective combination of a glycoprotein affecting protease and a sputum-degrading agent.

2. The method of claim 1, wherein the glycoprotein affecting protease is a cysteine protease.

3. The method of claim 1, wherein the glycoprotein affecting protease is selected from one or more of the group consisting of: bromelain, papain, ficain, actinidain, zingibain, fastuosain and ananain.

4. The method of claim 1, wherein the sputum-degrading agent comprises a disulphide bond breaking agent.

5. The method of claim 4, wherein the disulphide bond breaking agent is acetylcysteine, nacystelyn, N-acystelyn, cysteamine, erdosteine, s-carboxymethylcysteine, glutathione, dithiothreitol, mercapto-ethanesulphonate, carbocysteine, dornase alfa, gelsolin, thymosin P4, dextran, dithiobutylamine (DTBA) and heparin.

6. The method of claim 1, wherein the sputum-degrading agent comprises one or more of the group consisting of: a mucolytic agent, DNase, a PNAG degrading agent, serratiopeptidase, amylase, hyaluronidase and elastase.

7. The method claim 1, wherein the glycoprotein affecting protease and sputum-degrading agent are administered simultaneously or sequentially.

8. The method of claim 1, wherein the glycoprotein affecting protease and sputum-degrading agent are nebulized before administration.

9. The method of claim 1, wherein the glycoprotein affecting protease and sputum-degrading agent are provided in the form of a powder for inhalation from a dry powder inhaler.

10. The method of claim 1, wherein one or more additional therapeutic agents are co-administered to the patient with the combination.

11. The method of claim 10, wherein the one or more additional therapeutic agents are selected from the group consisting of antiviral agents, antibacterial agents, bronchodilators and expectorants.

12. The method of claim 1, wherein the respiratory disease is selected from the group consisting of cystic fibrosis, chronic obstructive pulmonary disease (COPD), acute respiratory distress syndrome (ARDS), bronchiectasis, ventilator associated pneumonia, asthma, sputum retention, mucus plugging, ciliary disfunction such as primary ciliary dyskinesia and bacterial infections.

13. The method of claim 1 where the respiratory disease is not a viral infection.

14. The method of claim 1 where the respiratory disease is not a bacterial infection involving biofilm.

15. A method for treating a respiratory disease in a patient, the method comprising directly instilling a therapeutically effective combination of a glycoprotein affecting protease and a sputum-degrading agent into the patient's airway.

16. The method of claim 15, wherein the combination is directly instilled into the trachea, bronchi or lower airway of the patient.

17. The method of claim 15, wherein the combination is instilled by a direct injection to the site of the disease or mucus plug in the airway at the time of a bronchoscopy.

18. A method for treating a disease or condition in a patient, the method comprising administering a therapeutically effective amount of a glycoprotein affecting protease to the patient via inhalation.

19. A method for inhibiting cytokine activity in a patient or for reducing an inflammatory response in a patient, the method comprising administering via inhalation a therapeutically effective combination of a glycoprotein affecting protease and a sputum-degrading agent.

20-32. (canceled)
